(19) United States
(12) Patent Application Publication
(10) Pub. No.: US 2025/0281654 A1
(43) Pub. Date: Sep. 11, 2025

(54) METHODS FOR TREATING CANCER USING COMBINATIONS OF EPIGENETIC THERAPIES AND RADIOCONJUGATE TARGETING AGENTS

(71) Applicant: ACTINIUM PHARMACEUTICALS, INC., New York, NY (US)

(72) Inventors: Dale L. LUDWIG, Rockaway, NJ (US); Paul DIAMOND, Fort Lee, NJ (US)

(21) Appl. No.: 18/036,723

(22) PCT Filed: Nov. 26, 2021
(86) PCT No.: PCT/US2021/060903

§ 371 Date: May 12, 2023

Related U.S. Application Data
(60) Provisional application No. 63/118,181, filed on Nov. 25, 2020.

(51) Int. Cl. A61K 51/10; A61K 31/167; A61K 31/18; A61K 31/4045; A61K 38/15

(52) U.S. Cl. CPC A61K 51/1027; A61K 31/167; A61K 31/18; A61K 31/4045; A61K 38/15; A61K 51/1096

(57) ABSTRACT

The invention provides methods for treating a cancer, such as acute myeloid leukemia, in a mammalian subject that include administering to the subject (i) an epigenetic drug such as one or both an HDAC inhibitor and an LSD1/KDM1A inhibitor, and (ii) a radioisotope-labeled agent that targets cancer cells in the subject, wherein the amounts of the epigenetic drug(s) and radiolabeled agent, when administered in conjunction with one another, are therapeutically effective.

### CROSS-REFERENCE TO RELATED APPLICATIONS

This application claims priority to U.S. provisional application Ser. No. 63/118,181 filed Nov. 25, 2020 which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Oct. 20, 2023, is named REPLACEMENT_10202023.txt and is 184,125 bytes in size.

### FIELD OF THE INVENTION

The present invention relates to the field of radiotherapeutics.

### BACKGROUND OF THE INVENTION

### HDAC Inhibitors

Histone deacetylases (“HDACs”) are a class of enzymes that remove acetyl groups from an epsilon N-acetyl lysine residue on a histone, thereby permitting the histone to wrap DNA more tightly. This action is opposite to the function of histone acetyltransferase. Tightly wrapping DNA is important because DNA expression is regulated by acetylation and de-acetylation.

Histone tails are normally positively charged due to the amine groups present on their lysine and arginine amino acids. These positively charged residues help the histone tails interact with, and bind to, the negatively charged DNA backbone.

Acetylation, which occurs normally, changes amines into amides. This change neutralizes the positive charges on the histone, thereby decreasing the ability of the affected histone to bind to DNA. This, in turn, permits chromatin expansion and, thus, genetic transcription to occur. HDACs remove acetyl groups, thus increasing the positive charge of the histone tails, thus increasing the binding affinity between histones and DNA, thus condensing DNA structure, and thus preventing transcription.

HDACs are involved in a number of biochemical pathways relating, for example, to cell growth and death, and to human diseases such as cancer. As such, HDAC inhibitors are of interest as anti-cancer therapeutics.

To date, the FDA has approved four HDAC inhibitor drugs (vorinostat, romidepsin, belinostat, and panobinostat) as anticancer agents. Vorinostat and romidepsin are approved for treating patients having cutaneous T-cell lymphoma. Belinostat is approved for treating patients having peripheral T-cell lymphoma. Panobinostat is approved for treating patients having multiple myeloma.

### LSD1/KDM1A Inhibitors

Lysine-specific histone demethylase 1A (LSD1) also known as lysine (K)-specific demethylase 1A (KDM1A), is encoded by the human KDM1A gene and upregulated in various solid tumor and hematological malignancies LSD1 is a flavin-dependent monoamine oxidase that can demethylate mono- and di-methylated lysines, specifically histone 3, lysines 4 and 9 (H3K4 and H3K9). LSD1 functions as a transcription co-repressor by demethylating H3K4me2/1 and shaping chromatin into a repressive conformation through complexes formed by LSD1 and other numerous proteins. LSD1 also functions as a demethylase of non-histone protein by minimizing the reaction of p53 and 53BP1, a tumor suppressor gene, by removing a methyl group from p53K370me2, thereby suppressing the role of p53. LSD1 upregulation stimulates tumorigenesis by regulating chromatin remodeling and aggregation. Further, LSD1 upregulation can affect the cell cycle of cancer cells resulting in the inhibition of the p53.

### Radiopharmaceuticals

The term “radiopharmaceutical” (also called a “radioisotope-labeled agent”) refers to a source of ionizing radiation (e.g., alpha and beta particles, and gamma rays) linked to a targeting agent such as an antibody, an antigen-binding antibody fragment, peptide, or a non-antibody scaffold. A source of ionizing radiation linked to an antibody is referred to as an “antibody radioconjugate” (ARC)”. The antibody portion of an ARC may, for example, be an intact (full-length) antibody or an antigen-binding antibody fragment.

Actinium-225 (Ac-225) is an ideal source of radiation for such purpose. Ac-225 (Linear Energy Transfer=6.83 MeV; T½=10 days; path length 40-80 um) causes clustered DNA lesions including single-strand breaks (SSBs) and double-strand breaks (DSBs). Even one alpha emission may be lethal to a tumor cell. Importantly, the radiopharmaceutical (e.g., ARC), once bound to a tumor cell, does not need to enter the cell to kill it. So, the process for effecting cell killing is far simpler than that required for an antibody-drug (chemo) conjugate. In addition, because Ac-225 can emit four alpha particles as it decays over its 10-day half-life, a bound radiopharmaceutical (e.g., ARC) can kill not only the target cell but also adjacent unbound tumor cells, including those that may be target antigen-negative. Importantly, the short path length of an alpha emitter limits the field of damage to immediately adjacent cells (i.e., as few as 2-6 cell diameters). As a result, normal tissue is significantly spared.

### SUMMARY OF THE INVENTION

In one aspect, the invention provides a method for treating a subject afflicted with cancer, including administering to the subject (i) an epigenetic therapy such as one or both of an HDAC inhibitor and an LSD1 inhibitor, in conjunction with (ii) a radioisotope-labeled agent that targets cancer cells in the subject, wherein the amounts of the HDAC inhibitor and labeled agent, when administered in conjunction with one another, are therapeutically effective.

In another aspect, the invention provides a method for treating a human subject afflicted with acute myeloid leukemia, including administering to the subject (i) an epigenetic therapy such as one or both of an HDAC inhibitor such as vorinostat, romidepsin, belinostat, and/or panobinostat in conjunction and an LSD1 inhibitor such as seclidemstat, TCP (tranylcypromine), ORY-1001 (iadademstat), GSK2879552 (GSK), INCB059872, IMG-7289 (bomedemstat), ORY-2001 (vafidemstat), and/or CC-90011; with (ii) a radiolabeled CD33 targeting agent such as ²²⁵Ac-labeled HuM195, wherein the amounts of the HDAC inhibitor and the radiolabeled CD33 targeting agent, when administered in conjunction with one another, are therapeutically effective.

In a further aspect, the invention provides a method for inducing the death of a cancer cell, including contacting the cell with (i) one or both of an HDAC inhibitor and an LSD1 inhibitor in conjunction with (ii) a radioisotope-labeled agent that targets the cancer cell, wherein the amounts of HDAC inhibitor and/or LSD1 inhibitor, and radiolabeled targeting agent, when contacted with the cell in conjunction with one another, are effective to induce the cell's death.

In still another aspect, the invention provides a method for inducing the death of an acute myeloid leukemia (AML) cell, including contacting the cell with (i) one or both of an HDAC inhibitor such as vorinostat, romidepsin, belinostat, or panobinostat and an LSD1 inhibitor such as seclidemstat, TCP (tranylcypromine), ORY-1001 (iadademstat), GSK2879552 (GSK), INCB059872, IMG-7289 (bomedemstat), ORY-2001 (vafidemstat), and/or CC-90011 in conjunction with (ii) a radiolabeled CD33 targeting agent such as ²²⁵Ac-labeled HuM195, wherein the amounts of HDAC inhibitor and/or LSD1 inhibitor, and ²²⁵Ac-labeled HuM195, when contacted with the cell in conjunction with one another, are effective to induce the cell's death.

In still further aspects, this invention provides two articles of manufacture. The first article of manufacture includes (i) one or both of an HDAC inhibitor (e.g., vorinostat, romidepsin, belinostat, or panobinostat) and an LSD1 inhibitor (e.g. seclidemstat, TCP (tranylcypromine), ORY-1001 (iadademstat), GSK2879552 (GSK), INCB059872, IMG-7289 (bomedemstat), ORY-2001 (vafidemstat), and/or CC-90011) and (ii) a label instructing the user (e.g., a healthcare provider) to treat a subject (e.g., a human) afflicted with a hematologic malignancy (e.g., acute myeloid leukemia or myelodysplastic syndrome) by administering the HDAC inhibitor and/or LSD1 inhibitor to the subject in conjunction with a radioisotope-labeled agent (e.g., a radiolabeled CD33 targeting agent such as ²²⁵Ac-labeled HuM195) that targets cancer cells in the subject, wherein the amounts of the HDAC inhibitor and/or LSD1 inhibitor, and radiolabeled targeting agent, when administered in conjunction with one another, are therapeutically effective.

The second article of manufacture provided includes (i) a radioisotope-labeled agent (e.g., ²²⁵Ac-labeled HuM195) that targets cancer cells and (ii) a label instructing the user (e.g., a healthcare provider) to treat a subject (e.g., a human) afflicted with a hematologic malignancy (e.g., acute myeloid leukemia or myelodysplastic syndrome) by administering the labeled agent to the subject in conjunction with an HDAC inhibitor (e.g., vorinostat, romidepsin, belinostat, and/or panobinostat) and/or an LSD1 inhibitor (e.g., seclidemstat, TCP (tranylcypromine), ORY-1001 (iadademstat), GSK2879552 (GSK), INCB059872, IMG-7289 (bomedemstat), ORY-2001 (vafidemstat), and/or CC-90011), wherein the amounts of the HDAC inhibitor and/or LSD1 inhibitor, and radiolabeled agent, when administered in conjunction with one another, are therapeutically effective. Additional features, advantages, and embodiments of the invention may be set forth or apparent from consideration of the following detailed description, drawings if any, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

FIG. 1 shows a schematic diagram of the expression plasmids for HuM195. The humanized VL and VH exons of HuM195 are flanked by XbaI sites. The VL exon was inserted into mammalian expression vector pVk, and the VH exon into pVg1 (Co, et al., J. Immunol. 148:1149-1154, 1992).

FIG. 2 shows the complete sequence of the HuM195 light chain gene cloned in pVk between the XbaI and BamHI sites. The nucleotide number indicates its position in the plasmid pVk-HuM195. The VL and CK exons are translated in single letter code; the dot indicates the translation termination codon. The mature light chain begins at the double-underlined aspartic acid (D). The intron sequence is in italics. The polyA signal is underlined. The full-length nucleotide sequence and the encoded amino acid sequence are disclosed as SEQ ID NO:114 and SEQ ID NO:115 respectively.

FIG. 3 shows the complete sequence of the HuM195 heavy chain gene cloned in pVg1 between the XbaI and BamHI sites. The nucleotide number indicates its position in the plasmid pVg1-HuM195. The VH, CH1, H, CH2 and CH3 exons are translated in single letter code; the dot indicates the translation termination codon. The mature heavy chain begins at the double-underlined glutamine (Q). The intron sequences are in italics. The polyA signal is underlined. The full-length nucleotide sequence and the encoded amino acid sequence are disclosed as SEQ ID NO:116 and SEQ ID NO:117 respectively.

FIG. 4 shows the structure of ²²⁵Ac-Lintuzumab (²²⁵Ac-HuM195).

FIG. 5 shows a flowchart for the production of ²²⁵Ac-HuM195.

FIG. 6 shows a dosing protocol for ²²⁵Ac-Lintuzumab (²²⁵Ac-HuM195) treatment of AML, without HDACi.

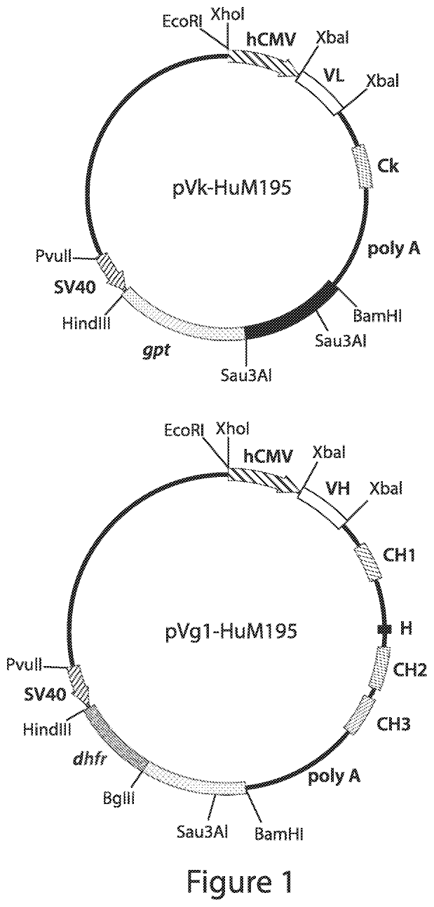

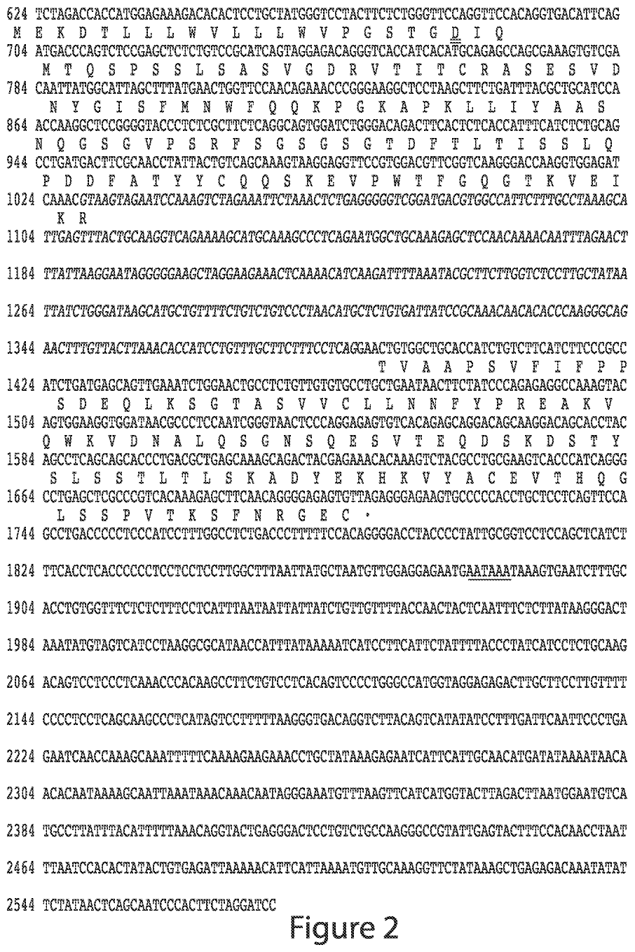

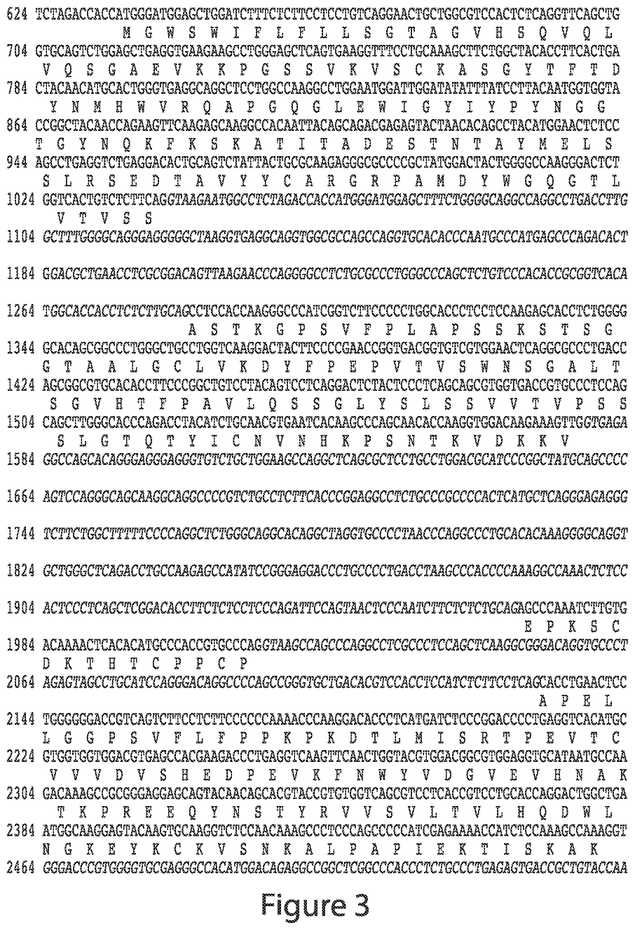

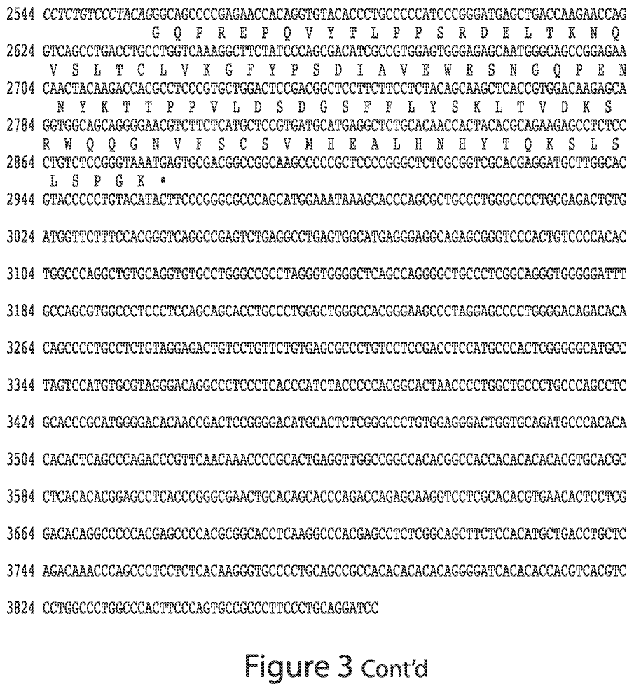

### DETAILED DESCRIPTION OF THE INVENTION

The invention provides combination therapies and related methods for treating cancer in a mammalian subject, such as a human patient, using one or both of an HDAC inhibitor and an LSD1 inhibitor, and a radioisotope-labeled cancer targeting-agent, such as but not limited to ²²⁵Ac-labeled HuM195.

### Definitions

In this disclosure, certain terms are used which shall have the meanings set forth as follows.

As used herein, “administer”, with respect to an agent, means to deliver the agent to a subject's body via any known method. Specific modes of administration include, without limitation, intravenous, intramuscular, oral, sublingual, transdermal, subcutaneous, intraperitoneal, intrathecal and intra-tumoral administration. HDAC inhibitors and LSD1 inhibitors may, for example, be administered orally or via injection, and radiolabeled targeting agents, such as antibody radioconjugates, may, for example, be administered intravenously.

In addition, in this invention, the various HDAC inhibitors, antibodies and other antigen-targeting agents used can be formulated using one or more routinely used pharmaceutically acceptable carriers/excipients. Such carriers are well known to those skilled in the art. For example, injectable drug delivery systems include solutions, suspensions, gels, microspheres and polymeric injectables, and can include excipients such as solubility-altering agents (e.g., ethanol, propylene glycol and sucrose) and polymers (e.g., polycaprylactones and PLGA's). Likewise, oral delivery systems include, for example, tablets and capsules. These can contain excipients such as binders (e.g., hydroxypropylmethyl-cellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

As used herein, the term “agent”, whether in reference to an HDAC inhibitor or a radioisotope-labeled agent, can be any type of compound or composition useful for such purpose. Types of agents include, without limitation, antibodies, other protein-based drugs, peptides, nucleic acids, carbohydrates and small molecules drugs.

As used herein, the term “antibody” includes, without limitation, (a) an immunoglobulin molecule including two heavy chains and two light chains and which recognizes an antigen; (b) polyclonal and monoclonal immunoglobulin molecules; (c) monovalent and divalent antigen binding fragments thereof such as Fab and Fab₂ fragments, and (d) bi-specific forms and multi-specific thereof. Immunoglobulin molecules may derive from any of the commonly known classes, including but not limited to IgA, secretory IgA, IgG and IgM. IgG subclasses are also well known to those in the art and include, but are not limited to, human IgG1, IgG2, IgG3 and IgG4. Antibodies can be both naturally occurring and non-naturally occurring. Furthermore, without limitation antibodies include chimeric antibodies, wholly synthetic antibodies, single chain antibodies, and fragments thereof, nanobodies, minibodies, affibodies, and scFv molecules. Antibodies may be human, humanized, or nonhuman.

As used herein, an “anti-CD33 antibody” is an antibody such as a monoclonal antibody, that binds to any available epitope of CD33 such as human CD33. In one embodiment, the anti-CD33 antibody binds to the epitope recognized by monoclonal antibody HuM195.

A “hematologic malignancy”, also known as a blood cancer, is a cancer that originates in blood-forming tissue, such as the bone marrow or other cells of the immune system. Hematologic malignancies that may be treated according to the invention include, without limitation, leukemias, lymphomas, and related disorders such as such as acute myeloid leukemia (AML), acute promyelocytic leukemia, acute lymphoblastic leukemia (ALL), acute mixed lineage leukemia, chronic myeloid leukemia (CML), chronic lymphocytic leukemia (CLL), hairy cell leukemia and large granular lymphocytic leukemia, myelodysplastic syndrome (MDS), myeloproliferative disorders (polycythemia vera, essential thrombocytosis, primary myelofibrosis and chronic myeloid, leukemia, cutaneous T-cell lymphoma (CTCL), peripheral T-cell lymphoma (CTCL), and multiple myeloma. Hematologic malignancies are characterized by hematologic malignancy-associated antigens. Such antigen may, for example, be a protein and/or carbohydrate marker found exclusively or predominantly on the surface of a cancer cell associated with that particular malignancy. Examples of hematologic malignancy-associated antigens that may be targeted include, without limitation, CD20, CD33, CD38, CD45, CD52, CD123 and CD319.

The antibody “HuM195” (also known as lintuzumab) is known, as are methods of making it. Likewise, methods of labeling HuM195 with ²²⁵Ac are known. These methods are exemplified, for example, in Scheinberg, et al., U.S. Pat. No. 6,683,162. This information is also exemplified in the examples and figures below.

As used herein, administering to a subject an HDAC inhibitor and/or an LSD1 inhibitor “in conjunction with” a radioisotope-labeled agent that targets cancer cells in the subject means administering the HDAC inhibitor before, during and/or after administration of the radiolabeled agent. This administration includes, without limitation, the following scenarios: (i) the epigenetic therapy(ies) is administered first, and the labeled agent is administered second; (ii) the H epigenetic therapy(ies) is administered concurrently with the labeled agent (e.g., the HDAC inhibitor is administered orally once per day for n days, and the labeled agent is administered intravenously in a single dose on one of days 2 through n−1 of the epigenetic therapy(ies) regimen); (iii) the epigenetic therapy(ies) is administered concurrently with the labeled agent (e.g., the epigenetic therapy(ies) is administered orally for a duration of greater than one month (e.g., orally once per day for 35 days, 42 days, 49 days, or a longer period during which the cancer being treated does not progress and during which the epigenetic therapy(ies) does not cause unacceptable toxicity), and the labeled agent is administered intravenously in a single dose on a day within the first month of the epigenetic therapy(ies) regimen); and (iv) the labeled agent is administered first (e.g., intravenously in a single dose or a plurality of doses over a period of weeks), and the epigenetic therapy(ies) is administered second (e.g., orally once per day for 21 days, 28 days, 35 days, 42 days, 49 days, or a longer period during which the cancer being treated does not progress and during which the epigenetic therapy(ies) does not cause unacceptable toxicity. Additional permutations are provided below in the Examples section. Those skilled in the art will recognize that when two or more therapeutic agents are administered in sufficient temporal proximity, the activity and/or the effect of each agent can temporally overlap with the other(s) in the treated subject.

As used herein, “HDAC” (i.e., histone deacetylase) includes, without limitation, all HDAC classes, such as class I, class IIA, class IIB, and class IV. Also included, without limitation, are all members of these HDAC classes, such as HDAC1, HDAC2, HDAC3, HDAC4, HDAC5, HDAC6, HDAC7, HDAC8, HDAC9, HDAC10, and HDAC11.

As used herein, “HDAC inhibitor”, also referred to as HDACi, includes, without limitation, FDA-approved HDAC inhibitors and experimental HDAC inhibitors. Without limitation, HDAC inhibitors that may be used in the various embodiments of this invention include vorinostat, romidepsin, belinostat, panobinostat, tucidinostat, pracinostat, CG 200745, entinostat, tacedinaline, mocetinostat, abexinostat, givinostat, resminostat, quisinostat, mocetinostat, and rocilinostat, as well others disclosed herein.

As used herein, “inducing” the death of a cancer cell includes, without limitation, (i) directly causing the cell's death, and (ii) indirectly causing the cell's death (e.g., by triggering a cascade of biochemical events that ultimately leads to the cell's death).

As used herein, an “LSD1 inhibitor” includes any agent that inhibits the lysine-specific histone demethylase activity of LSD1. Without limitation, LSD1 inhibitors that may be used in the various embodiments of the invention include seclidemstat, TCP (tranylcypromine), ORY-1001 (iadademstat), GSK2879552 (GSK), INCB059872 (Imago BioSciences), IMG-7289 (bomedemstat; Imago BioSciences), ORY-2001 (vafidemstat), and CC-90011 (Celgene), as well as others disclosed herein.

As used herein, “myelodysplastic syndrome”, also known as MDS, is a disorder that occurs when blood-forming cells in the bone marrow become abnormal (e.g., dysplastic), leading to low numbers of one or more types of blood cells. An example of a myelodysplastic syndrome cell is a mutated myeloid precursor cell lacking the ability to differentiate.

As used herein, a “radioisotope” can be an alpha-emitting isotope, a beta-emitting isotope, and/or a gamma-emitting isotope. Accordingly, as used herein, “radioisotope” is synonymous with “radionuclide.” Examples of radioisotopes that may be used in connection with the various embodiment sof the invention include without limitation the following: ⁹⁰Y, ⁸⁹Sr, ¹⁵³Sm, ³²P, ²²⁵Ac, ²¹³Bi, ²¹³Po, ²¹¹At, ²¹²Bi, ²¹³Bi, ²²³Ra, ²²⁷Th, ¹⁴⁹Tb, ¹³¹I, ¹³⁷Cs, ²¹²Pb, ¹⁰³Pd, ¹⁶⁶Ho, ¹⁸⁶Re, ¹⁸⁸Re, ⁶⁷Cu, ¹⁹⁹Au, ¹⁰⁵Rh, ²¹¹As, and ¹⁷⁷Lu. Thus, the radiolabeled antibodies envisioned in this invention include, without limitation, ⁹⁰Y-HuM195, ⁸⁹Sr-HuM195, ¹⁵³Sm-HuM195, ³²P-HuM195, ²²⁵Ac-HuM195, ²¹³Bi-HuM195, ²¹³Po-HuM195, ²¹¹At-HuM195, ²¹²Bi-HuM195, ²¹³Bi-HuM195, ²²³Ra-HuM195, ²²⁷Th-HuM195, ¹⁴⁹Tb-HuM195, ¹³¹I-HuM195, ¹³⁷Cs-HuM195, ²¹²Pb-HuM195, ¹⁰³Pd-HuM195, and ¹⁷⁷Lu-HuM195. Each of the antibody radioconjugates above is also envisioned, mutatis mutandis, and without limitation, for each of the following antibodies: alemtuzumab (Campath®), ibritumomab tiuxetan (Zevalin©), brentuximab vedotin (Adcetris®), trastuzumab (Herceptin®), trastuzumab emtansine (Kadcyla©), gemtuzumab ozogamicin (Mylotarg®), B1836858, BC8/apamistamab, daratumumab (Darzalex®), felzartamab/MOR202, isatuximab/SAR650984 (Sarclisa®), TAK-169, AV-203, CDX-3379, HMBD 001, patritumab, seribantumab, elgemtumab/LJM716, lumretuzumab/RG7116/RO5479599, GSK2849330, mapatumumab, tigatuzumab/CS-1008, drozitumab, lexatumumab, conatumumab, MED10641, PF-06263507/A1mcMMAF, Anti-5T4 SYD1875, ASN004, AVA-020, Tb535, Hu3S193, B3, IGN311, BR96/BMS-182248-01, girentuximab, BAY 794620, BAY 2701439, MEDI-4276, XMT-1522, zolbetuximab, TST001, A315, labetuzumab, sacituzumab govitecan-hziy (Trodelvy™), SAR566658, DMOT4039, anetumab ravtansine, BAY 2287411, and amatuximab. Methods for affixing a radioisotope to an antibody or other targeting agent (i.e., labeling the antibody or other targeting agent with a radioisotope) are well known in the art.

As used herein, the term “subject” includes, without limitation, a mammal such as a human, a non-human primate, a dog, a cat, a horse, a sheep, a goat, a cow, a rabbit, a pig, a rat and a mouse. Where the subject is human, the subject can be of any age. For example, the subject can be 60 years or older, 65 or older, 70 or older, 75 or older, 80 or older, 85 or older, or 90 or older. Alternatively, the subject can be 50 years or younger, 45 or younger, 40 or younger, 35 or younger, 30 or younger, 25 or younger, or 20 or younger. For a human subject afflicted with AML or MDS, the subject can be newly diagnosed, or relapsed and/or refractory, or in remission.

As used herein, “targeting agent” means a synthetic or biological molecule that preferentially and/or specifically binds a target molecule, such as a cancer-associated antigen or otherwise preferentially accumulates in cancer cells or and/or tumor tissue, in a subject and which may, for example, be radiolabeled for the delivery of therapeutic amounts of ionizing radiation to cancer cells within the subject. Without limitation, suitable types of targeting agents for use in the invention include antibodies such as IgG₁, IgG₂, IgG₃ and IgG₄, antigen-binding antibody fragments such as Fab and Fab₂, peptides such as GRPR-binding peptides such as RM-2, non-antibody antigen binding scaffolds/antibody mimetics, such as DARPins, and small molecules, such as PSMA-binding small molecules such as DUPA.

As used herein, an amount of HDAC inhibitor and/or LSD1 inhibitor, and an amount of radioisotope-labeled agent that targets cancer cells in the subject, when administered in conjunction with each other, are “therapeutically effective” if the subject is treated.

As used herein, “treating” a subject afflicted with a disorder shall include, without limitation, (i) slowing, stopping or reversing the disorder's progression, (ii) slowing, stopping or reversing the progression of the disorder's symptoms, (iii) reducing the likelihood of the disorder's recurrence, and/or (iv) reducing the likelihood that the disorder's symptoms will recur. In one embodiment, treating a subject afflicted with a disorder means (i) reversing the disorder's progression, ideally to the point of eliminating the disorder, and/or (ii) reversing the progression of the disorder's symptoms, ideally to the point of eliminating the symptoms, and/or (iii) reducing or eliminating the likelihood of relapse (i.e., consolidation, which is a common goal of post-remission therapy for AML and, ideally, results in the destruction of any remaining leukemia cells).

The treatment of hematologic malignancy, such as the treatment of AML, can be measured according to a number of clinical endpoints. These include, without limitation, survival time (such as weeks, months or years of improved survival time, e.g., one, two or more months' of additional survival time), and response status (such as complete remission (CR), complete remission with incomplete platelet recovery (CRp), complete remission with incomplete peripheral blood recovery (CRi), morphologic leukemia-free state (MLFS) and partial remission (PR)).

In one embodiment, treatment of hematologic malignancy, such as the treatment of AML, can be measured in terms of remission. Included here are the following non-limiting examples. (1) Morphologic complete remission (“CR”): ANC≥1,000/mcl, platelet count 100,000/mcl, <5% bone marrow blasts, no Auer rods, no evidence of extramedullary disease. (No requirements for marrow cellularity, hemoglobin concentration). (2) Morphologic complete remission with incomplete blood count recovery (“CRi”): Same as CR but ANC may be <1,000/mcl and/or platelet count<100,000/mcl. (3) Partial remission (PR): ANC 1,000/mcl, platelet count>100,000/mcl, and at least a 50% decrease in the percentage of marrow aspirate blasts to 5-25%, or marrow blasts <5% with persistent Auer rods. These criteria and others are known, and are described, for example, in SWOG Oncology Research Professional (ORP) Manual Volume I, Chapter 11A, Leukemia (2014).

### Embodiments of the Invention

This invention combines the use of different agents to treat cancers and proliferative disorders such as pre-cancerous conditions. Here, one or both of an HDAC inhibitor and LSD1 inhibitor, are administered in conjunction with a radiolabeled targeting agent such as antibody radioconjugate to more effectively treat patients with solid tumors or hematologic malignancies.

Specifically, this invention provides a method for treating a mammalian subject afflicted with cancer or proliferative disorder, such as human patient, including administering to the subject (i) an HDAC inhibitor and/or an LSD1 inhibitor, in conjunction with (ii) a radioisotope-labeled agent that targets cancer cells in the subject, wherein the amounts of the HDAC inhibitor and/or LSD1 inhibitor, and radiolabeled agent, when administered in conjunction with one another, are therapeutically effective.

The cancer treated may, for example, be a solid tumor, for example, breast cancer, HER2-positive breast cancer, HER2-negative breast cancer, HER3-positive breast cancer, tamoxifen-resistant breast cancer, triple negative breast cancer (TNBC), ovarian cancer, prostate cancer, castration-resistant prostate cancer (CRPC) lung cancer, small cell lung cancer (SCLC), non-small cell lung cancer (NSCLC), squamous cell carcinoma of the head and neck, gastric cancer, colorectal cancer, pancreatic cancer, brain cancer (e.g., glioblastoma and neuroblastoma), liver cancer (e.g. hepatocellular carcinoma or cholangiocarcinoma), sarcoma or melanoma. The cancer may be metastatic or non-metastatic.

The cancer treated may, for example, be a hematologic malignancy, for example, a CD33-expressing hematological malignancy, a myeloid malignancy, multiple myeloma, acute myeloid leukemia, or myelodysplastic syndrome.

The radioisotope-labeled targeting agent may, for example, be a radioisotope labeled CD33 targeting agent such as a radiolabeled anti-CD33 monoclonal antibody, for example, radioisotope labeled lintuzumab (HuM195), gemtuzumab, or vadastuximab, or a CD33-binding fragment of one of the aforementioned antibodies.

The radioisotope may, for example, be selected from the group consisting of ⁹⁰Y, ⁸⁹Sr, ¹⁵³Sm, ³²P, ²²⁵Ac, ²¹³Bi, ²¹³Po, ²¹¹At, ²¹²Bi, ²¹³Bi, ²²³Ra, ²²⁷Th, ¹⁴⁹Tb, ¹³¹I, ¹³⁷Cs, ²¹²Pb ¹⁰³Pd ¹⁶⁶Ho, ¹⁸⁶Re, ¹⁸⁸Re, ⁶⁷Cu, ¹⁹⁹Au, ¹⁰⁵Rh, ²¹¹As, and ¹⁷⁷Lu.

In a specific embodiment, the radioisotope-labeled agent is ²²⁵Ac-labeled HuM195 (also referred to herein as ²²⁵Ac-HuM195).

In another specific embodiment, the radioisotope-labeled agent is ¹⁷⁷Lu-labeled HuM195 (¹⁷⁷Lu-HuM195).

In the various embodiments of this invention involving an HDAC inhibitor, the HDAC inhibitor may be any agent performing that function. For example, the HDAC inhibitor may be vorinostat, romidepsin, belinostat, panobinostat, tucidinostat, pracinostat, CG 200745, entinostat, tacedinaline, mocetinostat, abexinostat, givinostat, resminostat, quisinostat, mocetinostat, rocilinostat, JB-802 (Jubilant Therapeutics Inc.), or JBI-295 (Jubilant Therapeutics Inc.).

An HDAC inhibitor that may be used may, for example, be a PROTAC (proteolysis-targeting chimera) agent such as any of the following compounds or a pharmaceutically acceptable salt thereof:

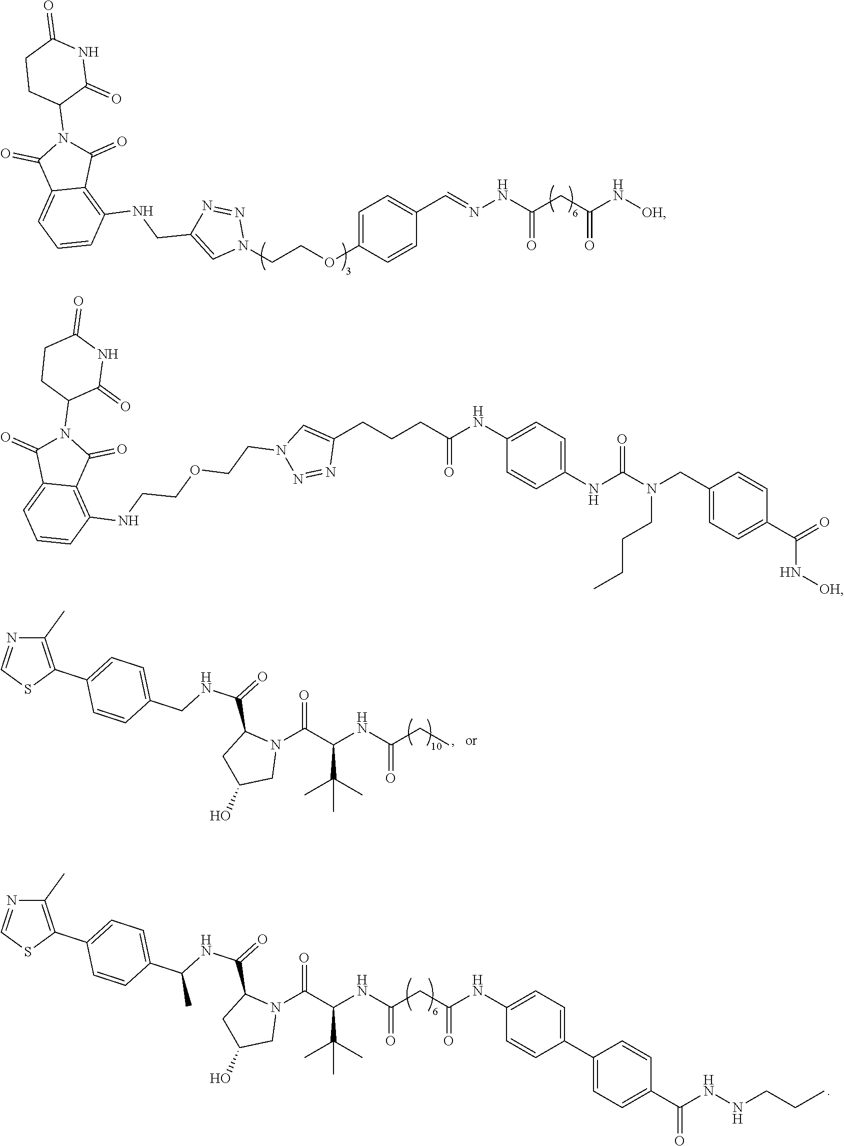

In the various embodiments of this invention involving an LSD1 inhibitor, the LSD1 inhibitor may be any agent performing that function, such as seclidemstat, TCP (tranylcypromine), ORY-1001 (iadademstat), GSK2879552 (GSK), INCB059872 (Imago BioSciences), IMG-7289 (Bomedemstat; Imago BioSciences), ORY-2001 (Vafidemstat), and CC-90011 (Celgene).

An HDAC inhibitor that may be used may, for example, be an agent having one or more additional activities.

An HDAC inhibitor that may be used may also be a LSD1 inhibitor, such as any of the following dual inhibitors or a pharmaceutically acceptable salt thereof:

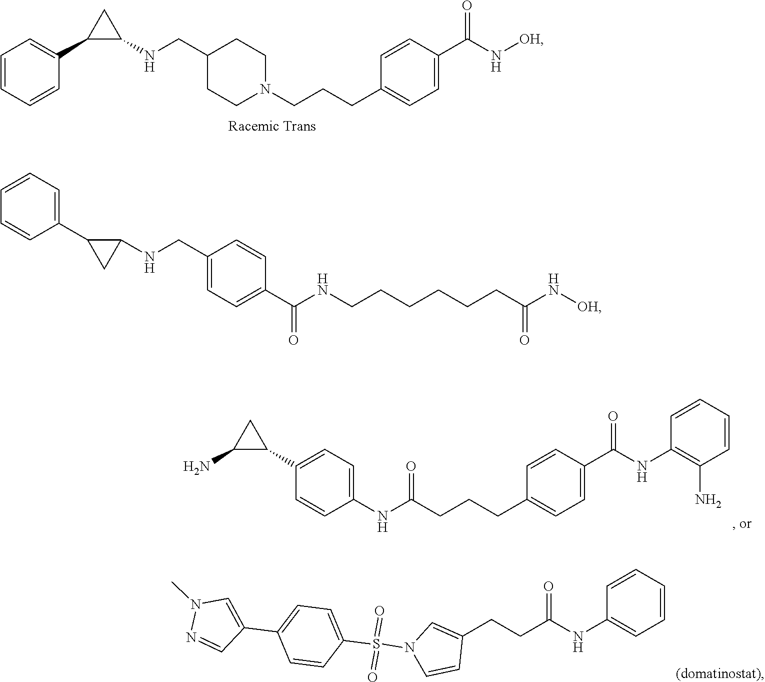 [SMILES: O=C(NO)c1ccc(CCCN2CCC(CN[C@H]3C[C@@H]3c3ccccc3)CC2)cc1 ; O=C(CCCCCCNC(=O)c1ccc(CNC2CC2c2ccccc2)cc1)NO ; Nc1ccccc1NC(=O)c1ccc(CCCC(=O)Nc2ccc([C@H]3C[C@@H]3N)cc2)cc1 ; Cn1cc(-c2ccc(S(=O)(=O)n3ccc(CCC(=O)Nc4ccccc4)c3)cc2)cn1]

or
any of the dual LSD1/HDAC inhibitors disclosed in U.S. Pub. No. 20200308110.

An HDAC inhibitor that may be used may also be a kinase inhibitor, such as any of the following dual inhibitors or a pharmaceutically acceptable salt thereof:

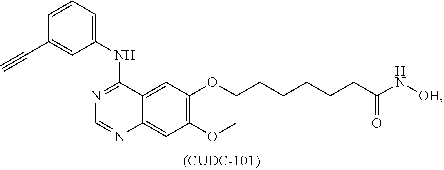 [SMILES: C#Cc1cccc(Nc2ncnc3cc(OC)c(OCCCCCCC(=O)NO)cc23)c1]

a dual HDAC/EGFR inhibitor, or

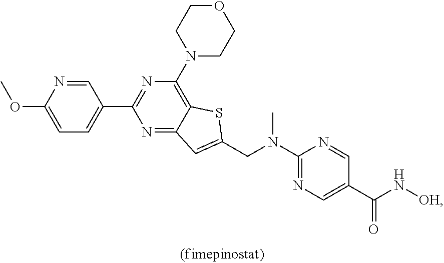 [SMILES: COc1ccc(-c2nc(N3CCOCC3)c3sc(CN(C)c4ncc(C(=O)NO)cn4)cc3n2)cn1]

a dual PI3K/HDAC inhibitor.

An HDAC inhibitor that may be used may also have DNA-alkylating activity, such as any of the following dual activity agents or a pharmaceutically acceptable salt thereof:

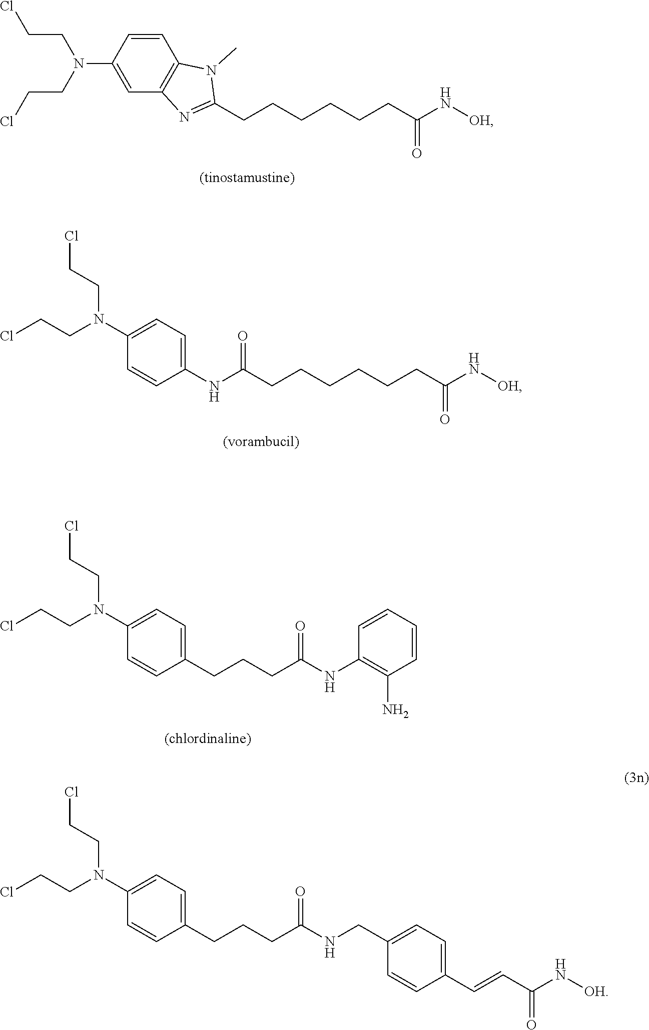 [SMILES: Cn1c(CCCCCCC(=O)NO)nc2cc(N(CCCl)CCCl)ccc21 ; O=C(CCCCCCC(=O)Nc1ccc(N(CCCl)CCCl)cc1)NO ; Nc1ccccc1NC(=O)CCCc1ccc(N(CCCl)CCCl)cc1 ; O=C(/C=C/c1ccc(CNC(=O)CCCc2ccc(N(CCCl)CCCl)cc2)cc1)NO]

An HDAC inhibitor that may be used may also be a topoisomerase inhibitor, such as the following dual inhibitor or a pharmaceutically acceptable salt thereof:

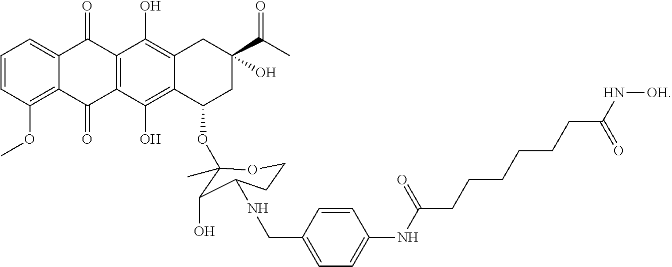 [SMILES: COc1cccc2c1C(=O)c1c(O)c3c(c(O)c1C2=O)C[C@@](O)(C(C)=O)C[C@@H]3OC1(C)OCCC(NCc2ccc(NC(=O)CCCCCCC(=O)NO)cc2)C1O]

An HDAC inhibitor that may be used may also be a BET protein (BRD2, BRD3, BRD4, BRDT) inhibitor, such as any of the following dual inhibitors or a pharmaceutically acceptable salt thereof:

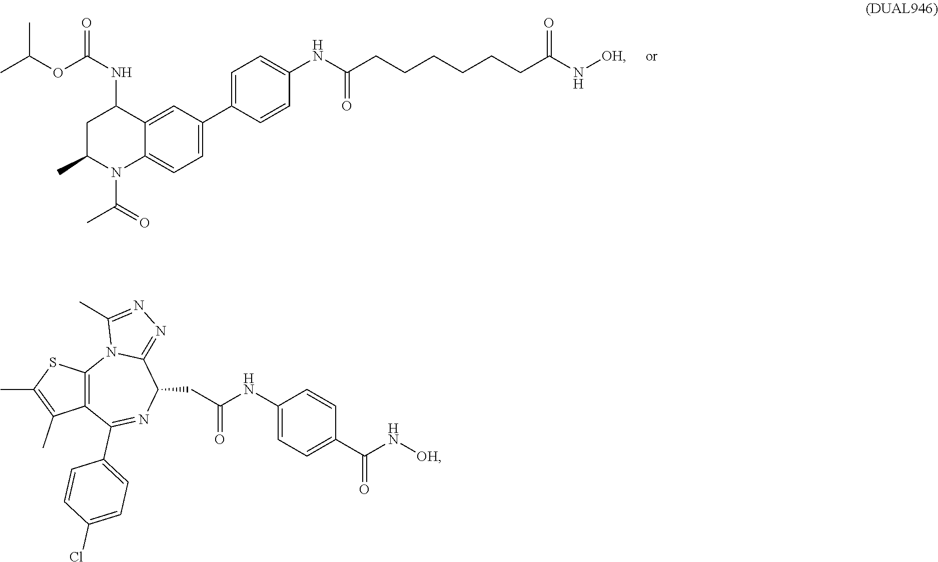 [SMILES: CC(=O)N1c2ccc(-c3ccc(NC(=O)CCCCCCC(=O)NO)cc3)cc2C(NC(=O)OC(C)C)C[C@@H]1C ; Cc1sc2c(c1C)C(c1ccc(Cl)cc1)=N[C@@H](CC(=O)Nc1ccc(C(=O)NO)cc1)c1nnc(C)n1-2]

An HDAC inhibitor that may be used may also be a heat shock protein 90 (Hsp90) inhibitor, such as any of the following dual inhibitors or a pharmaceutically acceptable salt thereof:

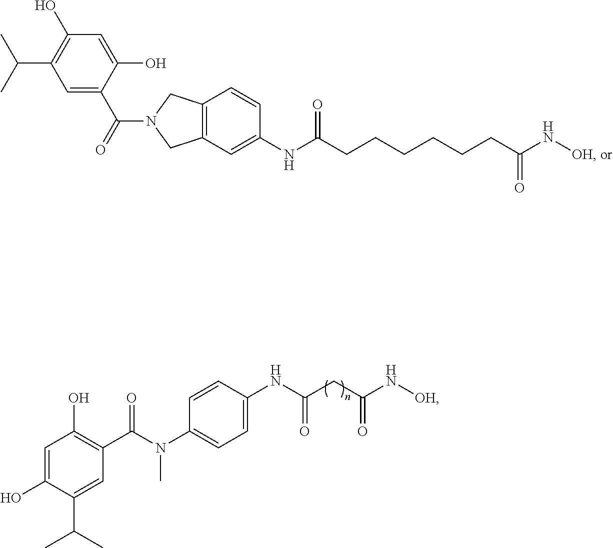

for example, where n is 1, 2, 3, 4, 5, 6, 7, 8, 9, or 10.

In the various embodiments of this invention, the radioisotope-labeled agent may be any agent, such as any known in the art, that targets cancer cells, for example, one approved by the FDA.

The radioisotope labeled targeting agent may, for example, preferentially and/or specifically bind to one or more of the following antigens, such as to the human forms of the following antigens: mesothelin, TSHR, CD19, CD123, CD22, CD30, CD45, CD171, CD138, CS-1, CLL-1, GD2, GD3, B-cell maturation antigen (BCMA), Tn Ag, prostate specific membrane antigen (PSMA), GRPR, ROR1, FLT3, TROP2, T-cell receptor gamma (TCRγ) chain alternate reading frame protein (TRAP), fibroblast activation protein (FAP), calreticulin, phosphatidylserine, GRP78 (BiP), TAG72, CD38, CD44v6, CEA, EPCAM, B7H3, KIT, IL-13Ra2, interleukin-11 receptor a (IL-IIRa), PSCA, PRSS21, VEGFR2, LewisY, CD24, platelet-derived growth factor receptor-beta (PDGFR-beta), SSEA-4, CD20, Folate receptor alpha (FRa), ERBB2 (Her2/neu), Her3, MUCI, epidermal growth factor receptor (EGFR), EGFRvIII, NCAM, Prostase, PAP, ELF2M, Ephrin B2, IGF-I receptor, CAIX, LMP2, gplOO, bcr-abl, tyrosinase, EphA2, Fucosyl GM1, sLe, GM3, DR5, 5T4, TGS5, HMWMAA, o-acetyl-GD2, Folate receptor beta, TEM1/CD248, TEM7R, CLDN6, GPRC5D, CXORF61, CD97, CD 179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-la, MAGE-A1, legumain, HPV E6,E7, MAGE AI, MAGEA3, MAGEA3/A6, ETV6-AML, sperm protein 17, XAGE1, Tie 2, MAD-CT-1, MAD-CT-2, Fos-related antigen 1, prostein, survivin and telomerase, PCTA-I/Galectin 8, KRAS, MelanA/MARTI, Ras mutant, hTERT, sarcoma translocation breakpoints, ML-IAP, ERG (TMPRSS2 ETS fusion gene), NA17, PAX3, Androgen receptor, Cyclin B I, MYCN, RhoC, TRP-2, CYP1B 1, BORIS, SART3, PAX5, OY-TES 1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, GPA7, and IGLL1.

Exemplary DR5 (death receptor 5) targeting agents that may be radiolabeled for use in the invention include the monoclonal anti-DR5 antibodies mapatumumab, conatumumab, lexatumumab, tigatuzumab, drozitumab, and LBY-135. Such DR5 targeting agents may, for example, be used in combination with an HDAC inhibitor and/or LSD1 inhibitor for the treatment of ovarian, breast, cervical prostate, gastric, bladder, lung, melanoma, colorectal and squamous cell carcinoma cancers and any of the cancers disclosed herein.

Exemplary 5T4 (Trophoblast glycoprotein (TBPG)) targeting agents that may be radiolabeled for use in the invention include the anti-5T4 monoclonal antibodies MED10641, ALG.APV-527, Tb535, H6-DM5, and ZV0508, as well as Naptumomab estafenatox or the Fab portion thereof. Such 5T4 targeting agents may, for example, be used in combination with an HDAC inhibitor and/or LSD1 inhibitor for the treatment of ovarian, head and neck, breast, prostate, gastric, bladder, lung, melanoma, colorectal and squamous cell carcinoma cancers and any of the cancers disclosed herein.

Exemplary HER2 (ERBB2) targeting agents that may be radiolabeled for use in the invention include the monoclonal antibodies trastuzumab and pertuzumab. Applicants have successfully conjugated Trastuzumab with p-SCN-DOTA and radiolabeled the composition with 225Ac or 177Lu. The anti-HER2 antibody may, for example, also be a multi-specific antibody, such as bispecific antibody, against any available epitope of HER3/HER2 such as MM-111 and MM-141/Istiratumab from Merrimack Pharmaceuticals, MCLA-128 from Merus NV, and MEHD7945A/Duligotumab from Genentech. Radiolabeled HER2 targeting agents may, for example, be used in combination with an HDAC inhibitor and/or LSD1 inhibitor for treatment of HER2-expressing cancers such as ovarian, breast, metastatic breast, esophageal, lung, cervical, and endometrial cancers including but not limited to those that are both HER2- and HER3-positive.

In one embodiment, the radioisotope-labeled agent is an antibody that specifically binds to a antigen selected from the group consisting of CD33, CD45, CD38, Her3, DR5 (TRAIL-R2), 5T4, Lewis-Y, CAIX, Her2, Claudin 18.2, CEACAM5, MUC1, and mesothelin, such as the human forms of these antigens.

In another embodiment, the radioisotope-labeled targeting agent is an antibody selected from the group consisting of alemtuzumab, ibritumomab, ibritumomab tiuxetan, brentuximab, brentuximab vedotin, trastuzumab, trastuzumab emtansine, gemtuzumab, gemtuzumab ozogamicin, lintuzumab, B1836858, BC8/apamistamab, daratumumab, felzartamab/MOR202, isatuximab/SAR650984, TAK-169, AV-203, CDX-3379, HMBD 001, patritumab, seribantumab, elgemtumab/LJM716, lumretuzumab/RG7116/RO5479599, GSK2849330, mapatumumab, tigatuzumab/CS-1008, drozitumab, lexatumumab, conatumumab, MED10641, PF-06263507/A1mcMMAF, Anti-5T4 SYD1875, ASN004, AVA-020, Tb535, Hu3S193, B3, IGN311, BR96/BMS-182248-01, girentuximab, BAY 794620, BAY 2701439, MEDI-4276, XMT-1522, zolbetuximab, TST001, A315, labetuzumab, labetuzumab govitecan, SAR566658, DMOT4039, anetumab, anetumab ravtansine, BAY 2287411, and amatuximab, or an antigen-binding fragment of one of said antibodies.

The radioisotope-labeled targeting agent may, for example, be an anti-CD33 antibody, such as an anti-CD33 monoclonal antibody, radiolabeled with an isotope selected from the group consisting of ⁹⁰Y, ⁸⁹Sr, ¹⁵³Sm, ³²P, ²²⁵Ac, ²¹³Bi, ²¹³Po, ²¹¹At, ²¹²Bi, ²¹³Bi, ²²³Ra, ²²⁷Th, ¹⁴⁹Tb, ¹³¹I, ¹³⁷CS ²¹²Pb, ¹⁰³Pd, and ¹⁷⁷Lu. For example, the radiolabeled anti-CD33 antibody may be ²²⁵Ac-labeled HuM195 or ¹⁷⁷Lu-labeled HuM195.

This invention further provides a method for treating a mammalian subject, such as a human subject, afflicted with a myeloid proliferative disorder such as acute myeloid leukemia or myelodysplastic syndrome, including administering to the subject (i) one or both of an HDAC inhibitor such as vorinostat, romidepsin, belinostat, panobinostat or any disclosed herein, and an LSD1 inhibitor, such as seclidemstat, TCP (tranylcypromine), ORY-1001 (iadademstat), GSK2879552 (GSK), INCB059872, IMG-7289 (bomedemstat), ORY-2001 (vafidemstat), CC-90011) or any disclosed herein, in conjunction with (ii) ²²⁵Ac-labeled HuM195, wherein the amounts of the HDAC inhibitor and/or LSD1 inhibitor, and ²²⁵Ac-labeled HuM195, when administered in conjunction with one another, are therapeutically effective. Specific exemplifications envisioned in this method are the following combinations: (i) ²²⁵Ac-labeled HuM195 and vorinostat; (ii) ²²⁵Ac-labeled HuM195 and romidepsin; (iii) ²²⁵Ac-labeled HuM195 and belinostat; and (iv) ²²⁵Ac-labeled HuM195 and panobinostat.

This invention further provides a method for treating a human subject afflicted with myelodysplastic syndrome, including administering to the subject (i) an HDAC inhibitor selected from the group consisting of vorinostat, romidepsin, belinostat, and panobinostat in conjunction with (ii) ²²⁵Ac-labeled HuM195, wherein the amounts of the HDAC inhibitor and ²²⁵Ac-labeled HuM195, when administered in conjunction with one another, are therapeutically effective. Specifically envisioned in this method are the following combinations: (i) ²²⁵Ac-labeled HuM195 and vorinostat; (ii) ²²⁵Ac-labeled HuM195 and romidepsin; (iii) ²²⁵Ac-labeled HuM195 and belinostat; and (iv) ²²⁵Ac-labeled HuM195 and panobinostat.

This invention provides a method for inducing the death of a mammalian cancer cell, including contacting the cell with (i) an HDAC inhibitor in conjunction with (ii) a radioisotope-labeled agent that targets the cancer cell, wherein the amounts of HDAC inhibitor and radiolabeled agent, when contacted with the cell in conjunction with one another, are effective to induce the cell's death. The cancer cell may, for example, be a human cancer cell.

In one embodiment of this method, the cancer cell is a tumor cell selected from the group consisting of a breast cancer cell, an ovarian cancer cell, a prostate cancer cell, a lung cancer cell, a squamous cell carcinoma of the head and neck cell, a gastric cancer cell, a pancreatic cancer cell, a brain cancer cell, a liver cancer cell, a sarcoma cell, and a melanoma cell.

In another embodiment of this method, the cancer cell is a hematologic cancer cell, such as a multiple myeloma cell, an acute myeloid leukemia cell or a myelodysplastic syndrome cell (e.g., a mutated myeloid precursor cell lacking the ability to differentiate).

The radioisotope-labeled targeting agent may, for example, specifically binds to a cancer-associated antigen selected from the group consisting of CD33, CD45, CD38, Her3, DR5 (TRAIL-R2), 5T4, Lewis-Y, CAIX, Her2, Her3, Claudin 18.2, CEACAM5, MUC1, and mesothelin. Such targeting agent may, for example, be an antibody, an antigen-binding antibody fragment, an antibody mimetic, a peptide, a ligand, or a small molecule.

In another embodiment, the radioisotope-labeled agent includes or is the antibody which is, or the antibody portion of, one of the following: alemtuzumab, ibritumomab, ibritumomab tiuxetan, brentuximab, brentuximab vedotin, trastuzumab, trastuzumab emtansine, gemtuzumab, gemtuzumab ozogamicin, lintuzumab, B1836858, BC8/apamistamab, daratumumab, felzartamab/MOR202, isatuximab/SAR650984, TAK-169, AV-203, CDX-3379, HMBD 001, patritumab, seribantumab, elgemtumab/LJM716, lumretuzumab/RG7116/RO5479599, GSK2849330, mapatumumab, tigatuzumab/CS-1008, drozitumab, lexatumumab, conatumumab, MED10641, PF-06263507/A1mcMMAF, Anti-5T4 SYD1875, ASN004, AVA-020, Tb535, Hu3S193, B3, IGN311, BR96/BMS-182248-01, girentuximab, BAY 794620, BAY 2701439, MEDI-4276, XMT-1522, zolbetuximab, TST001, A315, labetuzumab, labetuzumab govitecan, SAR566658, DMOT4039, anetumab, anetumab ravtansine, BAY 2287411, and amatuximab.

The amino acid sequences of the heavy chain and the light chain of Trastuzumab reported by DrugBank Online are: heavy chain (SEQ ID NO:102) and light chain (SEQ ID NO:103) and an anti-HER2 binding antibody including one or both of said chains may be embodied in or used in the various embodiments of the invention.

The amino acid sequences of the heavy chain and the light chain of Pertuzumab reported by DrugBank Online are: heavy chain (SEQ ID NO:104) and light chain (SEQ ID NO:105) and an anti-HER2 binding antibody including one or both of said chains may be embodied in or used in the various embodiments of the invention.

An exemplary HER3 antibody that may be radiolabeled and embodied in and/or used in the presently disclosed invention may, for example, include a murine monoclonal antibody against HER3 including a heavy chain having the amino acid sequence as set forth in SEQ ID NO:9 or 11 and/or a light chain having the amino acid sequence as set forth in SEQ ID NO:10 or 12, or an antibody such as a humanized antibody derived from one or more of said sequences. An exemplary HER3 antibody that may be radiolabeled and embodied in and/or used in the presently disclosed invention may include or a heavy chain with an N-terminal region having the sequence set forth in SEQ ID NO:13 and/or a light chain with an N-terminal region having the sequence as set forth in SEQ ID NO:14. A HER3 antibody that may be similarly embodied or used in various aspect of the invention may, for example, include the heavy chain variable region having the amino acid sequence as set forth in SEQ ID NO:7, and/or a light chain variable region having an amino acid sequence as set forth in SEQ ID NO:8, and/or a heavy chain including one or more of CDR1, CDR2 and CDR3 having the amino acid sequences respectively set forth in SEQ ID NOS:1-3, and/or a light chain with one or more of the CDR1, CD2 and CDR3 having the amino acid sequences respectively set forth in SEQ ID NOS:4-6. A HER3 antibody embodied in and/or used in any of the aspects of the invention may, for example, include any combination of the aforementioned light chain sequences and/or heavy chain sequences.

An exemplary HER3 antibody includes an immunoglobulin heavy chain variable region including a CDR-H1 including SEQ ID NO:15, a CDR-H2 including SEQ ID NO:16, and a CDR-H3 including SEQ ID NO:17, and/or an immunoglobulin light chain variable region including a CDR-L1 including SEQ ID NO:18, a CDR-L2 including SEQ ID NO:19, and a CDR-L3 including SEQ ID NO:20. An exemplary An exemplary HER3 antibody includes an immunoglobulin heavy chain variable region including SEQ ID NO:21 and/or an immunoglobulin light chain variable region including SEQ ID NO:22. An exemplary HER3 antibody includes an immunoglobulin heavy chain amino acid sequence of SEQ ID NO:23 and/or an immunoglobulin light chain amino acid sequence of SEQ ID NO:24.

An exemplary HER3 antibody includes an immunoglobulin heavy chain variable region including a CDR-H1 including SEQ ID NO:25, a CDR-H2 including SEQ ID NO:26, and a CDR-H3 including SEQ ID NO:27; and/or an immunoglobulin light chain variable region including a CDR-L1 including SEQ ID NO:28, a CDR-L2 including SEQ ID NO:29, and a CDR-L3 including SEQ ID NO:30. An exemplary HER3 antibody includes an immunoglobulin heavy chain variable region including SEQ ID NO:31 and/or an immunoglobulin light chain variable region including SEQ ID NO:32. An exemplary HER3 antibody includes an immunoglobulin heavy chain amino acid sequence of SEQ ID NO:33 and/or an immunoglobulin light chain amino acid sequence of SEQ ID NO:34.

An exemplary HER3 antibody includes an immunoglobulin heavy chain variable region including a CDR-H1 including SEQ ID NO:35, a CDR-H2 including SEQ ID NO:36, and a CDR-H3 including SEQ ID NO:37; and/or an immunoglobulin light chain variable region including a CDR-L1 including SEQ ID NO:38, a CDR-L2 including SEQ ID NO:39, and a CDR-L3 including SEQ ID NO:40. An exemplary HER3 antibody includes an immunoglobulin heavy chain variable region including SEQ ID NO:41, and/or an immunoglobulin light chain variable region SEQ ID NO:42. An exemplary HER3 antibody includes an immunoglobulin heavy chain amino acid sequence of SEQ ID NO:43 and an immunoglobulin light chain amino acid sequence of SEQ ID NO:44.

An exemplary HER3 antibody includes an immunoglobulin heavy chain variable region including a CDR-H1 including SEQ ID NO:45, a CDR-H2 including SEQ ID NO:46, and a CDR-H3 including SEQ ID NO:47; and/or an immunoglobulin light chain variable region including a CDR-L1 including SEQ ID NO:48, a CDR-L2 including SEQ ID NO:29, and a CDR-L3 including SEQ ID NO:49. An exemplary HER3 antibody includes an immunoglobulin heavy chain variable region including SEQ ID NO:50 and/or an immunoglobulin light chain variable region including SEQ ID NO:51. An exemplary HER3 antibody includes an immunoglobulin heavy chain amino acid sequence of SEQ ID NO:52 and/or an immunoglobulin light chain amino acid sequence of SEQ ID NO:53.

An exemplary HER3 antibody includes an immunoglobulin heavy chain variable region including a CDR-H1 including SEQ ID NO:54, a CDR-H2 including SEQ ID NO:55, and a CDR-H3 including SEQ ID NO:56; and/or an immunoglobulin light chain variable region including a CDR-L1 including SEQ ID NO:28, a CDR-L2 including SEQ ID NO:29, and a CDR-L3 including SEQ ID NO:30. An exemplary HER3 antibody includes an immunoglobulin heavy chain variable region including SEQ ID NO:57 and/or an immunoglobulin light chain variable region including SEQ ID NO:58. An exemplary HER3 antibody includes an immunoglobulin heavy chain amino acid sequence of SEQ ID NO:59 and/or an immunoglobulin light chain amino acid sequence of SEQ ID NO: 60.

An exemplary HER3 antibody includes an immunoglobulin heavy chain variable region including a CDR-H1 including SEQ ID NO:61, a CDR-H2 including SEQ ID NO:62, and a CDR-H3 including SEQ ID NO:63; and/or an immunoglobulin light chain variable region including a CDR-L1 including SEQ ID NO:64, a CDR-L2 including SEQ ID NO:65, and a CDR-L3 including SEQ ID NO:66. An exemplary HER3 antibody includes an immunoglobulin heavy chain variable region including SEQ ID NO:67, and/or an immunoglobulin light chain variable region including SEQ ID NO:68. An exemplary HER3 antibody includes an immunoglobulin heavy chain amino acid sequence of SEQ ID NO:69 and an immunoglobulin light chain amino acid sequence of SEQ ID NO:70.

An exemplary HER3 antibody includes an immunoglobulin heavy chain variable region including a CDR-H1 including SEQ ID NO:71, a CDR-H2 including SEQ ID NO:72, and a CDR-H3 including SEQ ID NO:66; and/or an immunoglobulin light chain variable region including a CDR-L1 including SEQ ID NO:28, a CDR-L2 including SEQ ID NO:29, and a CDR-L3 including SEQ ID NO:30. An exemplary HER3 antibody includes an immunoglobulin heavy chain variable region including SEQ ID NO:73, and/or an immunoglobulin light chain variable region including SEQ ID NO:74. An exemplary HER3 antibody includes an immunoglobulin heavy chain amino acid sequence of SEQ ID NO:75 and/or an immunoglobulin light chain amino acid sequence of SEQ ID NO:76.

An exemplary HER3 antibody includes an immunoglobulin heavy chain amino acid sequence of SEQ ID NO:77 and/or an immunoglobulin light chain amino acid sequence of SEQ ID NO:78.

An exemplary HER3 antibody includes an immunoglobulin light chain variable region including SEQ ID NOS:86, 87, 88, 89, 90 or 91 and/or a heavy chain variable region including SEQ ID NOS:79, 80, 81, 82, 83, 84 or 85.

An exemplary HER3 antibody includes an immunoglobulin heavy chain sequence including SEQ ID NO:92, 94, 95, 98 or 99 and/or an immunoglobulin light chain sequence including SEQ ID NO:93, 96, 97, 100 or 101.

Exemplary HER3 antibodies also include Barecetamab (ISU104) from Isu Abxis Co and any of the HER3 antibodies disclosed in U.S. Pat. No. 10,413,607.

Exemplary HER3 antibodies also include HMBD-001 (10D1F) from Hummingbird Bioscience Pte. and any of the HER3 antibodies disclosed in International Pub. Nos. WO 2019185164 and WO2019185878, U.S. Pat. No. 10,662,241; and U.S. Pub. Nos. 20190300624, 20210024651, and 20200308275.

Exemplary HER3 antibodies also include the HER2/HER3 bispecific antibody MCLA-128 (i.e., Zenocutuzumab) from Merus N.V.; and any of the HER3 antibodies, whether monospecific or multi-specific, disclosed in U.S. Pub. Nos. 20210206875, 20210155698, 20200102393, 20170058035, and 20170037145.

Exemplary HER3 antibodies also include the HER3 antibody Patritumab (U3-1287), an antibody including heavy chain sequence SEQ ID NO:106 and/or light chain sequence SEQ ID NO:107 which are reported chains of Patritumab, and any of the HER3 antibodies disclosed in U.S. Pat. Nos. 9,249,230 and 7,705,130 and International Pub. No. WO2007077028.

Exemplary HER3 antibodies also include the HER3 antibody MM-121 and any of the HER3 antibodies disclosed in U.S. Pat. No. 7,846,440 and International Pub. No. WO2008100624.Exemplary HER3 antibodies also include the EGFR/HER3 bispecific antibody DL1 and any of the HER3 antibodies, whether monospecific or multi-specific, disclosed in U.S. Pat. Nos. 9,327,035 and 8,597,652, U.S. Pub. No. 20140193414, and International Pub. No. WO2010108127.

Exemplary HER3 antibodies also include the HER2/HER3 bispecific antibody MM-111 and any of the HER3 antibodies, whether monospecific or multi-specific, disclosed in U.S. Pub. Nos. 20130183311 and 20090246206 and International Pub. Nos. WO2006091209 and WO2005117973.

According to certain aspects, the HER3 targeting agent includes an anti-HER3 antibody that binds to an epitope of HER3 recognized by Patritumab from Daiichi Sankyo, Seribantumab (MM-121) from Merrimack Pharmaceuticals, Lumretuzumab from Roche, Elgemtumab from Novartis, GSK2849330 from GlaxoSmithKline, CDX-3379 of Celldex Therapeutics, EV20 and MP-RM-1 from MediPharma, Barecetamab (ISU104) from Isu Abxis Co., HMBD-001 (10D1F) from Hummingbird Bioscience Pte., REGN1400 from Regeneron Pharmaceuticals, and/or AV-203 from AVEO Oncology. According to certain aspects, the anti-HER3 antibody is selected from one or more of Patritumab, Seribantumab or an antibody including heavy chain sequence SEQ ID NO:108 and/or light chain sequence SEQ ID NO:109 which are reported for Seribantumab, Lumretuzumab or an antibody including heavy chain sequence SEQ ID NO:110 and/or light chain sequence SEQ ID NO:111 which are reported for Lumretuzumab, Elgemtumab or an antibody including heavy chain sequence SEQ ID NO:112 and/or light chain sequence SEQ ID NO:113 which are reported for Elgemtumab, AV-203, CDX-3379, GSK2849330, EV20, MP-RM-1, ISU104, HMBD-001 (10D1F), and REGN1400.

It should be understood that wherever in this disclosure specific antibodies, specific antibody heavy chains and specific antibody light chains are disclosed, against any target, also intended to be disclosed for embodiment in or use in the various aspects of the invention are antibodies, such as but not limited to immunoglobulins, such as but not limited to IgG, that (i) include the heavy chain variable region of the disclosed antibody or heavy chain, (ii) include 1, 2 or 3 of the heavy chain CDRs (e.g., by Kabat definition) of the disclosed antibody or heavy chain, (iii) include the light chain variable region of the disclosed antibody or light chain, and/or (iv) include 1, 2 or 3 of the light chain CDRs (e.g., by Kabat definition) of the disclosed antibody or light chain. It should also be understood that wherever in this disclosure an antibody heavy chain or an antibody light chain is disclosed that includes an N-terminal leader sequence, also intended to be disclosed for embodiment in and use in the various aspects of the invention are corresponding heavy chains and corresponding light chains that lack the leader sequence.

In still further embodiments of the invention, the radiolabeled targeting agent used in combination or conjunction with the epigenetic therapy(ies) for the treatment of a cancer or proliferative disorder such as any of those disclosed herein in a mammal, such as a human, includes a phospholipid-based cancer targeting agent. In certain embodiments, the phospholipid-based cancer targeting agent includes any of the radioactive phospholipid metal chelates disclosed in U.S. Pub. No. 20200291049, incorporated by reference herein, such as but not limited to

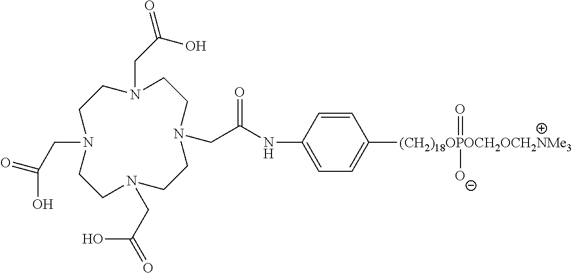

(a/k/a NM600) or a pharmaceutically acceptable salt thereof, chelated with a radionuclide, such as ²²⁵Ac, ¹⁷⁷Lu, or 90Y

In certain aspects, the lipid based radiolabeled targeting agent used with the epigenetic therapy includes any of the radiolabeled phospholipid compounds disclosed in U.S. Pub. No. 20140030187 or U.S. Pat. No. 6,417,384, each incorporated by reference herein, such as but not limited to

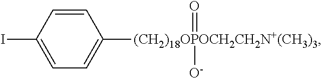

i.e., 18-(p-iodophenyl)octadecyl phosphocholine, wherein iodine is ¹³¹I (a/k/a NM404 I-131, and CLR 131), or a pharmaceutically acceptable salt thereof.

In certain aspects, the phospholipid-based radiolabeled targeting agent used with the epigenetic therapy(ies) includes any of the phospholipid drug conjugate compounds disclosed in U.S. Pat. No. 9,480,754, incorporated by reference herein.

In one embodiment, the radioisotope-labeled targeting agent is an anti-CD33 antibody, such as a monoclonal antibody or an antigen-binding fragment thereof, or an antibody mimetic, labeled with an isotope selected from the group consisting of ⁹⁰Y, ⁸⁹Sr, ¹⁵³Sm, ³²P, ²²⁵Ac, ²¹³Bi, ²¹³Po, ²¹¹At, ²¹²Bi, ²¹³Bi, ²²³Ra, ²²⁷Th, ¹⁴⁹Tb, ¹³¹I, ¹³⁷Cs, ²¹²Pb, ¹⁰³Pd, ¹⁶⁶Ho, ¹⁸⁶Re, ¹⁸⁸Re, ⁶⁷Cu, ¹⁹⁹Au, ¹⁰⁵Rh, ²¹¹As, and ¹⁷⁷Lu. For example, the radiolabeled anti-CD33 antibody may be ²²⁵Ac-labeled HuM195 or ¹⁷⁷Lu-labeled HuM195.

This invention further provides a method for inducing the death of a CD33-expressing hematological proliferative disorder cell such as an acute myeloid leukemia cell, a multiple myeloma cell, or a myelodysplastic syndrome cell, including contacting the cell with (i) one or both of an HDAC inhibitor such as any of those disclosed herein and an LSD1 inhibitor such as any of those disclosed herein, in conjunction with (ii) a radiolabeled CD33 targeting agent such as ²²⁵Ac-labeled HuM195, wherein the amounts of HDAC inhibitor and/or LSD1 inhibitor, and radiolabeled CD33 targeting agent, when contacted with the cell in conjunction with one another, are effective to induce the cell's death. Specific exemplifications envisioned in this method are the following combinations: (i) ²²⁵Ac-labeled HuM195 and vorinostat; (ii) ²²⁵Ac-labeled HuM195 and romidepsin; (iii) ²²⁵Ac-labeled HuM195 and belinostat; and (iv) ²²⁵Ac-labeled HuM195 and panobinostat.

This invention further provides a method for inducing the death of an acute myeloid leukemia cell, including contacting the cell with (i) one or both of an HDAC inhibitor such as any of those disclosed herein and an LSD1 inhibitor such as any of those disclosed herein, in conjunction with (ii) a radiolabeled CD33 targeting agent such as ²²⁵Ac-labeled HuM195, wherein the amounts of HDAC inhibitor and/or LSD1 inhibitor, and radiolabeled CD33 targeting agent, when contacted with the cell in conjunction with one another, are effective to induce the cell's death. Specific exemplifications envisioned in this method are the following combinations: (i) ²²⁵Ac-labeled HuM195 and vorinostat; (ii) ²²⁵Ac-labeled HuM195 and romidepsin; (iii) ²²⁵Ac-labeled HuM195 and belinostat; and (iv) ²²⁵Ac-labeled HuM195 and panobinostat.

This invention further provides a method for inducing the death of a myelodysplastic syndrome cell, including contacting the cell with (i) one or both of an HDAC inhibitor such as any of those disclosed herein and an LSD1 inhibitor such as any of those disclosed herein, in conjunction with (ii) a radiolabeled CD33 targeting agent such as ²²⁵Ac-labeled HuM195, wherein the amounts of HDAC inhibitor and/or LSD1 inhibitor, and radiolabeled CD33 targeting agent, when contacted with the cell in conjunction with one another, are effective to induce the cell's death. Specific exemplifications envisioned in this method are the following combinations: (i) ²²⁵Ac-labeled HuM195 and vorinostat; (ii) ²²⁵Ac-labeled HuM195 and romidepsin; (iii) ²²⁵Ac-labeled HuM195 and belinostat; and (iv) ²²⁵Ac-labeled HuM195 and panobinostat.

In addition to use in treating CD33-positive hematological proliferative disorders/cancers, radiolabeled CD33 targeting agents such as those disclosed herein may be used to treat a solid tumor cancer in a mammal by depleting CD33-positive myeloid-derived suppressor cells (MDSCs), in combination with treatment using one or both of an HDAC inhibitor such as any of those disclosed herein and an LSD1 inhibitor such as any of those disclosed herein. Accordingly, one embodiment of the invention provides a method for treating a solid tumor cancer such as any of those disclosed herein in a mammal, such as a human, that includes administering to the mammal: (i) one or both of an HDAC inhibitor such as any of those disclosed herein and an LSD1 inhibitor such as any of those disclosed herein, and (ii) a radiolabeled CD33 targeting agent such as ²²⁵Ac-labeled HuM195, wherein the amounts of HDAC inhibitor and/or LSD1 inhibitor, and radiolabeled CD33 targeting agent, when administered in conjunction with one another, are effective to treat the solid tumor cancer.

The HDAC inhibitor and/or LSD1 inhibitor may each, for example, be administered, such as daily or every other day, at a dose of 1.0 to 2,000 mg, or in any subrange bounded by integer values thereof, such as 1.0 to 1,000 mg, such as 25 to 1,000 mg, such as 10 to 500 mg. In one variation of the instant methods, each of the HDAC inhibitor and/or the LSD1 inhibitor is administered in doses and/or schedules provided with approved labeling or used in clinical trials of the agents. In one variation of the instant methods, the HDAC inhibitor, the LSD1 inhibitor, and/or the radiolabeled targeting agent, administered in doses that are less than, and/or in dosing regimens of shorter duration than, those presently prescribed on their respective labels or used in clinical trials of the agents. Embodiments of the invention in this regard are set forth in the examples section.

This invention also provides articles of manufacture. The first article includes (i) one or both of an HDAC inhibitor (e.g., vorinostat, romidepsin, belinostat, panobinostat and/or any of those disclosed herein) and an LSD1 inhibitor (e.g., seclidemstat, TCP (tranylcypromine), ORY-1001 (iadademstat), GSK2879552 (GSK), INCB059872, IMG-7289 (bomedemstat), ORY-2001 (vafidemstat), CC-90011, and/or any of those disclosed herein), and (ii) a label instructing the user (e.g., a healthcare provider) to treat a subject (e.g., a human) afflicted with a hematologic malignancy (e.g., acute myeloid leukemia or myelodysplastic syndrome) by administering the HDAC inhibitor to the subject in conjunction with a radioisotope-labeled agent (e.g., ²²⁵Ac-labeled HuM195) that targets cancer cells in the subject, wherein the amounts of the HDAC inhibitor and labeled agent, when administered in conjunction with one another, are therapeutically effective. Specific exemplifications envisioned in this article are the following combinations: (i) ²²⁵Ac-labeled HuM195 and vorinostat; (ii) ²²⁵Ac-labeled HuM195 and romidepsin; (iii) ²²⁵Ac-labeled HuM195 and belinostat; and/or (iv) ²²⁵Ac-labeled HuM195 and panobinostat.

The second article of manufacture includes (i) a radioisotope-labeled agent (e.g., ²²⁵Ac-labeled HuM195) that targets cancer cells and (ii) a label instructing the user (e.g., a healthcare provider) to treat a subject (e.g., a human) afflicted with a hematologic malignancy (e.g., acute myeloid leukemia, myelodysplastic syndrome, or multiple myeloma) by administering the labeled agent to the subject in conjunction with one or both of an HDAC inhibitor (e.g., vorinostat, romidepsin, belinostat, panobinostat and/or any of those disclosed herein) and an LSD1 inhibitor (e.g., seclidemstat, TCP (tranylcypromine), ORY-1001 (iadademstat), GSK2879552 (GSK), INCB059872, IMG-7289 (bomedemstat), ORY-2001 (vafidemstat), CC-90011, and/or any of those disclosed herein), wherein the amounts of the HDAC inhibitor and/or LSD1 inhibitor, and labeled agent, when administered in conjunction with one another, are therapeutically effective. Specific exemplifications envisioned in this article are the following combinations: (i) ²²⁵Ac-labeled HuM195 and vorinostat; (ii) ²²⁵Ac-labeled HuM195 and romidepsin; (iii) ²²⁵Ac-labeled HuM195 and belinostat; and/or (iv) ²²⁵Ac-labeled HuM195 and panobinostat.

Those skilled in the art will appreciate that methods for radiolabeling a targeting agent, directly or via a chelator group are well established.

Exemplary chelators that may be linked to targeting agents in the various aspects of the invention include: 1,4,7,10-tetraazacyclododecane-1,4,7-triacetic acid (DO3A) or a derivative thereof; 1,4,7-triazacyclononane-1,4-diacetic acid (NODA) or a derivative thereof; 1,4,7-triazacyclononane-1,4,7-triacetic acid (NOTA) or a derivative thereof; 1,4,7,10-tetraazacyclododecane-1,4,7,10-tetraacetic acid (DOTA) or a derivative thereof; 1,4,7-triazacyclononane, 1-glutaric acid-4,7-diacetic acid (NODAGA) or a derivative thereof; 1,4,7,10-tetraazacyclodecane, 1-glutaric acid-4,7,10-triacetic acid (DOTAGA) or a derivative thereof; 1,4,8,11-tetraazacyclotetradecane-1,4,8,11-tetraacetic acid (TETA) or a derivative thereof; 1,4,8,11-tetraazabicyclo[6.6.2]hexadecane-4,11-diacetic acid (CB-TE2A) or a derivative thereof; diethylene triamine pentaacetic acid (DTPA), its diester, or a derivative thereof; 2-cyclohexyl diethylene triamine pentaacetic acid (CHX-A″-DTPA) or a derivative thereof; deforoxamine (DFO) or a derivative thereof; 1,2-[[6-carboxypyridin-2-yl]methylamino]ethane (H₂dedpa) or a derivative thereof; DADA or a derivative thereof; 1,4,7,10-Tetraazacyclododecane-1,4,7,10-tetra(methylene phosphonic acid) (DOTP) or a derivative thereof; 4-amino-6-[[16-[(6-carboxypyridin-2-yl)methyl]-1,4,10,13-tetraoxa-7,16-diazacyclooctadec-7-yl]methyl]pyridine-2-carboxylic acid (MACROPA-NH₂) or a derivative thereof; MACROPA or a derivative thereof; 1,4,7,10-tetrakis(carbamoylmethyl)-1,4,7,10-tetraazacyclododecane (TCMC) or a derivative thereof; {4-[2-(bis-carboxymethylamino)-ethyl]-7-carboxymethyl-[1,4,7]triazonan-1-yI}-acetic acid (NETA) or a derivative thereof; Diamsar or a derivative thereof; 1,4,7-triazacyclononane-1,4,7-tris[methyl(2-carboxyethyl)phosphinic acid (TRAP, PRP9, TRAP-Pr) or a derivative thereof; N,N′-bis(6-carboxy-2-pyridylmethyl)ethylenediamine-N,N′-diacetic acid (H4octapa) or a derivative thereof; N,N′-[1-benzyl-1,2,3-triazole-4-yl]methyl-N,N′-[6-(carboxy)pyridin-2-yl]-1,2-diaminoethane (H2azapa) or a derivative thereof; N,N″-[[6-(carboxy)pyridin-2-yl]methyl]diethylenetriamine-N,N′,N″-triacetic acid (H5decapa) or a derivative thereof; N,N′-bis(2-hydroxy-5-sulfobenzyl)ethylenediamine-N,N′-diacetic acid (SHBED) or a derivative thereof; N,N′-bis(2-hydroxybenzyl)ethylenediamine-N,N′-diacetic acid (HBED) or a derivative thereof; 3,6,9,15-tetraazabicyclo[9.3.1]pentadeca-1(15),11,13-triene-3,6,9,-triacetic acid (PCTA) or a derivative thereof; desferrioxamine B (DFO) or a derivative thereof; N,N′-(methylenephosphonate)-N,N′-[6-(methoxycarbonyl)pyridin-2-yl]methyl-1,2-diaminoethane (H6phospa) or a derivative thereof; 1,4,7,10,13,16-hexaazacyclohexadecane-N,N′,N″,N″′,N″″,N″″′-hexaacetic acid (HEHA) or a derivative thereof; 1,4,7,10,13-pentaazacyclopentadecane-N,N′,N″,N″′,N″ ″-pentaacetic acid (PEPA) or a derivative thereof; or 3,4,3-LI(1,2-HOPO) or a derivative thereof.

According to certain aspects, the targeting agent may be radiolabeled through chemical conjugation of suitable bifunctional chelators that can chelate one or more radionuclides. Exemplary chelator molecules that may be used include p-SCN-Bn-DOTA, NH₂-DOTA, NH₂—(CH₂)₁₋₂₀-DOTA, NH₂-(PEG)₁₋₂₀-DOTA, HS-DOTA, HS—(CH₂)₁₋₂₀-DOTA, HS-(PEG)₁₋₂₀-DOTA, dibromo-S—(CH₂)₁₋₂₀-DOTA, dibromo-S-(PEG)₁₋₂₀-DOTA, p-SCN-Bn-DOTP, NH₂-DOTP, NH₂—(CH₂)₁₋₂₀-DOTP, NH₂-(PEG)₁₋₂₀-DOTP, HS-DOTP, HS—(CH₂)₁₋₂₀-DOTP, HS-(PEG)₁₋₂₀-DOTP, dibromo-S—(CH₂)₁₋₂₀-DOTP, and dibromo-S-(PEG)₁₋₂₀-DOTP. The chelator molecules may, for example, be attached to a targeting agent through a linker molecule. Exemplary linker molecules include:

—CH₂(C₆H₄)NH₂ or —CH₂(C₆H₄)NH—X—Y,
wherein X is
—R₂—CH₂CH₂O(CH₂CH₂O)ₙCH₂CH₂—,
—R₂—CH₂CH₂NHC(O)CH₂CH₂O(CH₂CH₂O)ₙCH₂CH₂—,
—R₂—(CH₂)ₙCH₂—,
—R₂—CH₂CH₂NHC(O)(CH₂)ₙCH₂—,
—R₂—CH(C(O)R₃)CH₂—, wherein R₃ is —OH or a short peptide (1-20 amino acids),
—R₂—CH₂CH₂O(CH₂CH₂O)ₙCH₂C(O)O—, or
—R₂—CH₂CH₂NHC(O)CH₂CH₂O(CH₂CH₂O)ₙCH₂CC(O)O—,
wherein n is 1-20, and
R₂ is —C(O)— or —C(S)NH—; and
Y is —NH₂ or —SR₄—, wherein R₄ is —H or —CH₂-3,5-bis(bromomethyl)benzene.

Targeting agents, such as protein targeting agents, for example antibodies and antigen-binding antibody fragments, and peptide targeting agents may, for example, be conjugated with a chelator for radiolabeling the targeting agent via chelation of a radionuclide. Such protein or peptide targeting agents, for example, that include lysine(s), may conveniently be conjugated to a DOTA chelating moiety using the bifunctional agent S-2-(4-Isothiocyanatobenzyl)-1,4,7,10-tetraazacyclododecane tetraacetic acid a/k/a/“p-SCN-Bn-DOTA” (Catalog #B205; Macrocyclics, Inc., Plano, TX, USA). p-SCN-Bn-DOTA may be synthesized by a multi-step organic synthesis fully described in U.S. Pat. No. 4,923,985. Chelation of a radionuclide by the DOTA moiety may be performed prior to chemical conjugation of the antibody with p-SCN-Bn-DOTA and/or after said conjugation.

Targeting agents containing one or more cysteine residues, such as peptides, proteins, antibodies and protein antibody mimetics that have one or more cysteines may, for example, be chemically conjugated to any of the chelator-bearing, such as DOTA-bearing, stable linkers disclosed in U.S. Pat. No. 11,000,604 titled “Reagent for site-selective bioconjugation of proteins or antibodies” for radionuclide labeling.

According to certain aspects of the present invention, administering a radiolabeled targeting agent to a subject in any of the embodiments may, for example, include administration of a composition that includes a radiolabeled fraction and a non-radiolabeled fraction of the targeting agent, such as an antibody, antibody fragment, etc. For example, the non-radiolabeled-labeled fraction may include the same antibody against the same epitope as the labeled fraction. In this way, the total radioactivity of the antibody may be varied or may be held constant while the overall antibody protein concentration may be held constant or may be varied, respectively. For example, the total protein concentration of non-radiolabeled labeled antibody fraction administered may be selected depending on the exact nature of the disease to be treated, age and weight of the patient, identity of the antibody, and the label (e.g., radionuclide) selected for labeling of the antibody. The composition may, for example, include the targeting agent in a ratio of radiolabeled. non-radiolabeled targeting agent of from about 0.01:10 to 1:1, such as 0.1:10 to 1:1 radiolabeled: non-radiolabeled. Various aspects of this invention will be better understood by reference to the following examples.

### EXAMPLES

### Example 1—Structure of ²²⁵Ac-Lintuzumab (²²⁵Ac-HuM195)

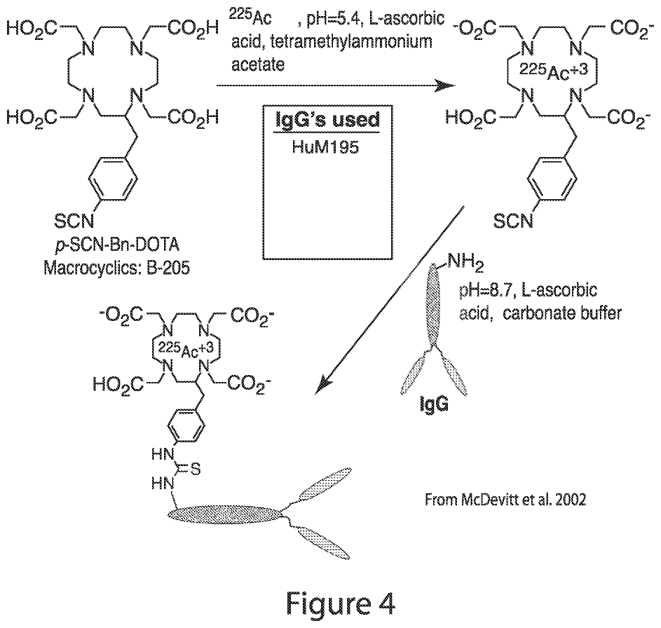

²²⁵Ac-Lintuzumab includes three key components; humanized monoclonal antibody HuM195 (generic name, lintuzumab), the alpha-emitting radioisotope ²²⁵Ac, and the bi-functional chelate 2-(p-isothiocyanatobenzyl)-1,4,7,10-tetraazacyclododecane-1,4,7,10-tetraacetic acid (p-SCN-Bn-DOTA). As depicted in FIG. 4, HuM195 is radiolabeled using the bi-functional chelate p-SCN-Bn-DOTA that binds to ²²⁵Ac and that is covalently attached to the IgG via a lysine residue on the antibody.

### Example 2—p-SCN-Bn-DOTA

DOTA, 2-(4-Isothiocyanatobenzyl)-1,4,7,10-tetraazacyclododecane tetraacetic acid (Macrocyclics item code B205-GMP) is synthesized by a multi-step organic synthesis that is fully described in U.S. Pat. No. 4,923,985.

### Example 3—Preparation of ²²⁵Ac-Lintuzumab (²²⁵Ac-HuM195)

The procedure for preparing ²²⁵Ac-Lintuzumab is based on the method described by Michael R. McDevitt, “Design and synthesis of ²²⁵Ac radioimmuno-pharmaceuticals, Applied Radiation and Isotope”, 57 (2002), 841-847. The procedure involves radiolabeling the bi-functional chelate, p-SCN-Bn-DOTA, with the radioisotope ²²⁵Ac, followed by binding of the radiolabeled p-SCN-Bn-DOTA to the antibody (HuM195). The construct, ²²⁵Ac-p-SCN-Bn-DOTA-HuM195, is purified using 10 DG size exclusion chromatography and eluted with 1% human serum albumin (HSA). The resulting drug product, Ac²²⁵-Lintuzumab, is then passed through a 0.2 μm sterilizing filter.

### Example 4—Process Flow for Preparation of ²²⁵Ac-Lintuzumab (²²⁵Ac-HuM195)

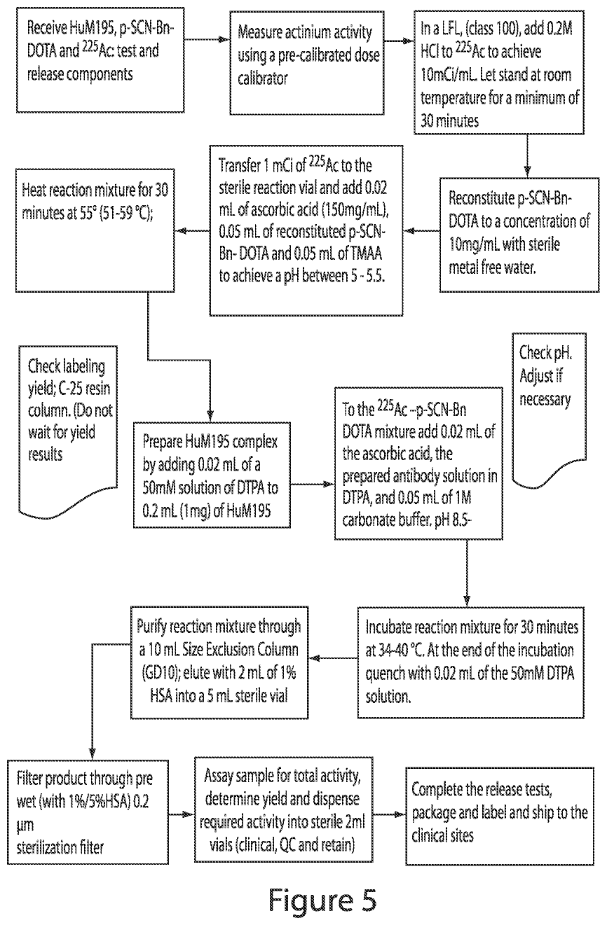

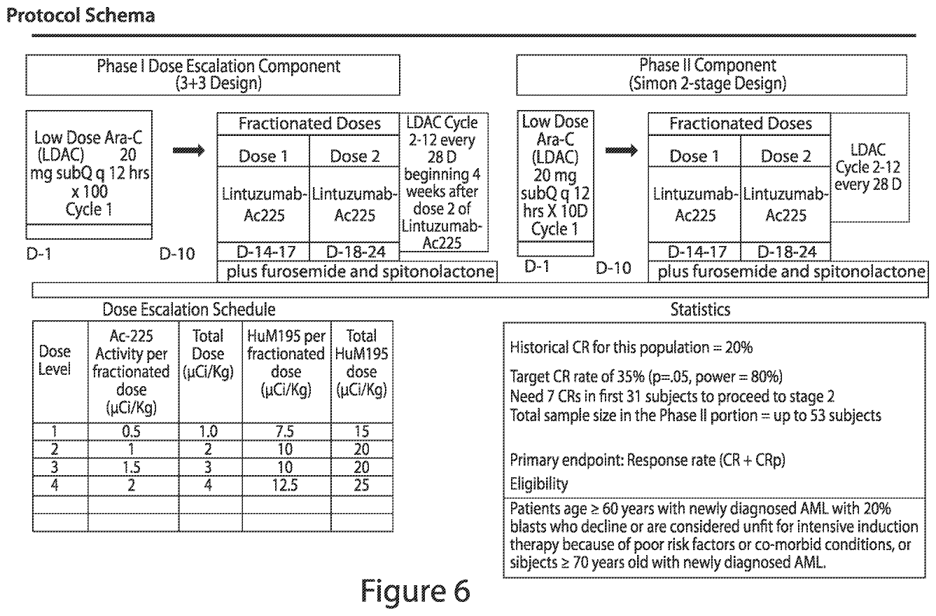

The procedure, shown in FIG. 5, begins with confirming the identity of all components and the subsequent QC release of the components to production.

The ²²⁵Ac is assayed to confirm the level of activity and is reconstituted to the desired activity concentration with hydrochloric acid. A vial of lyophilized p-SCN-Bn-DOTA is reconstituted with metal-free water to a concentration of 10 mg/mL. To the actinium reaction vial, 0.02 ml of ascorbic acid solution (150 mg/mL) and 0.05 ml of reconstituted p-SCN-Bn-DOTA are added and the pH adjusted to between 5 and 5.5 with 2M tetramethylammonium acetate (TMAA). The mixture is then heated at 55±4° C. for 30 minutes.

To determine the labeling efficiency of the ²²⁵Ac-p-SCN-Bn-DOTA, an aliquot of the reaction mixture is removed and applied to a 1 ml column of Sephadex C25 cation exchange resin. The product is eluted in 2-4 ml fractions with a 0.9% saline solution. The fraction of ²²⁵Ac activity that elutes is ²²⁵Ac-p-SCN-Bn-DOTA and the fraction that is retained on the column is un-chelated, unreactive ²²⁵Ac. Typically, the labeling efficiency is greater than 95%.

To the reaction mixture, 0.22 ml of previously prepared HuM195 in DTPA (1 mg HuM195) and 0.02 ml of ascorbic acid are added. The DTPA is added to bind any trace amounts of metals that may compete with the labeling of the antibody. The ascorbic acid is added as a radio-protectant. The pH is adjusted with carbonate buffer to pH 8.5-9. The mixture is heated at 37±3° C. for 30 minutes.

The final product is purified by size exclusion chromatography using 10DG resin and eluted with 2 ml of 1% HSA. Typical reaction yields are 10%.

### Example 5—Vorinostat—Normal and Reduced Dosing Regimens

Vorinostat is sold by Merck & Co., Inc. under the brand name Zolinza®. Vorinostat is sold in capsule form at 100 mg. The dosage is 400 mg taken orally once daily. Dosing continues for five consecutive days each week, and may be continued as long as there is no evidence of progressive disease or toxicity. This dosing regimen is referred to herein as the “normal” human dosing regimen for vorinostat, regardless of the disorder treated. Any dosing regimen having a shorter duration (e.g., four days or fewer per week) or involving the administration of less than 400 mg/day (e.g., 100 mg/day) is referred to herein as a “reduced” human dosing regimen. Examples of reduced human dosing regimens include the following: (i) 350 mg/day, 300 mg/day, 250 mg/day, 200 mg/day, 150 mg/day, 100 mg/day, 75 mg/day, 50 mg/day, 25 mg/day, and (ii)<400 mg/day; 300-350 mg/day, 250-300 mg/day, 200-250 mg/day, 150-200 mg/day, 100-150 mg/day, 75-100 mg/day, 50-75 mg/day, 25-50 mg/day, and <25 mg/day.

### Example 6—Romidepsin—Normal and Reduced Dosing Regimens

Romidepsin is sold by Gloucester Pharmaceuticals, Inc., under the brand name Istodax®. Romidepsin is sold as an injectable formulation. The dosage is 14 mg/m² administered intravenously over a four-hour period on days 1, 8, and 15 of a 28-day cycle. Cycles are repeated every 28 days provided that the patient continues to benefit from and tolerate the therapy. This dosing regimen is referred to herein as the “normal” human dosing regimen for romidepsin, regardless of the disorder treated. Any dosing regimen having a shorter duration (e.g., intravenous administration over a four-hour period only on days 1 and 8 of a 28-day cycle, or a limited number of cycles (e.g., a total of two 28-day cycles)) or involving delivering less than 14 mg/m² per administration (e.g., 10 mg/m² per administration) is referred to herein as a “reduced” human dosing regimen. Examples of reduced human dosing regimens include the following: (i) 13 mg/m² per administration, 12 mg/m² per administration, 11 mg/m² per administration, 10 mg/m² per administration, 9 mg/m² per administration, 8 mg/m² per administration, 7 mg/m² per administration, 6 mg/m² per administration, 5 mg/m² per administration, 4 mg/m² per administration, 3 mg/m² per administration, 2 mg/m² per administration, 1 mg/m² per administration, and 0.5 mg/m² per administration; (ii)<14 mg/m² per administration, 12-13 mg/m² per administration, 11-12 mg/m² per administration, 10-11 mg/m² per administration, 9-10 mg/m² per administration, 8-9 mg/m² per administration, 7-8 mg/m² per administration, 6-7 mg/m² per administration, 5-6 mg/m² per administration, 4-5 mg/m² per administration, 3-4 mg/m² per administration, 2-3 mg/m² per administration, 1-2 mg/m² per administration, 0.5-1 mg/m² per administration, <0.5 mg/m² per administration; and (iii) intravenous administration only on days 1 and 8 of a 28-day cycle, intravenous administration only on days 1 and 15 of a 28-day cycle, intravenous administration only on day 1 of a 28-day cycle, intravenous administration only on day 1 of a 21-day cycle, intravenous administration only on day 1 of a 14-day cycle; and intravenous administration only on day 1 of a 7-day cycle.

### Example 7—Belinostat—Normal and Reduced Dosing Regimens

Belinostat is sold by Spectrum Pharmaceuticals, Inc., under the brand name Beleodaq®. Belinostat is sold as an injectable formulation. The dosage is 1,000 mg/m² administered intravenously over a 30-minute period once daily on days 1-5 of a 21-day cycle. Cycles can be repeated until disease progression or unacceptable toxicity. This dosing regimen is referred to herein as the “normal” human dosing regimen for belinistat, regardless of the disorder treated. Any dosing regimen having a shorter duration (e.g., intravenous administration over a 30-minute period once daily only on days 1-3 of a 21-day cycle, or a limited number of cycles (e.g., a total of two 21-day cycles)) or involving delivering less than 1,000 mg/m² per administration (e.g., 500 mg/m² per administration) is referred to herein as a “reduced” human dosing regimen. Examples of reduced human dosing regimens include the following: (i) 900 mg/m² per administration, 800 mg/m² per administration, 700 mg/m² per administration, 600 mg/m² per administration, 500 mg/m² per administration, 400 mg/m² per administration, 300 mg/m² per administration, 250 mg/m² per administration, 200 mg/m² per administration, 150 mg/m² per administration, 100 mg/m² per administration, 75 mg/m² per administration, 50 mg/m² per administration, and 25 mg/m² per administration; (ii)<1,000 mg/m² per administration, 800-900 mg/m² per administration, 700-800 mg/m² per administration, 600-700 mg/m² per administration, 500-600 mg/m² per administration, 400-500 mg/m² per administration, 300-400 mg/m² per administration, 250-300 mg/m² per administration, 200-250 mg/m² per administration, 150-200 mg/m² per administration, 100-150 mg/m² per administration, 75-100 mg/m² per administration, 50-75 mg/m² per administration, 25-50 mg/m² per administration, and <25 mg/m² per administration; and (iii) intravenous administration only on days 1-4 of a 21-day cycle, intravenous administration only on days 1-3 of a 21-day cycle, intravenous administration only on days 1 and 2 of a 21-day cycle, intravenous administration only on day 1 of a 21-day cycle, intravenous administration only on day 1 of a 14-day cycle, and intravenous administration only on day 1 of a 7-day cycle.

### Example 8—Panobinostat—Normal and Reduced Dosing Regimens

Panobinostat is sold by Novartis Pharmaceuticals Corporation under the brand name Farydak®. Panobinostat is sold in capsule form at 10 mg, 15 mg, and 20 mg. The dosage is 20 mg taken orally once every other day for three doses per week (on days 1, 3, 5, 8, 10, and 12) of weeks one and two of each 21-day cycle. Dosing continues for eight 21-day cycles. This dosing regimen is referred to herein as the “normal” human dosing regimen for panobinostat, regardless of the disorder treated. Any dosing regimen having a shorter duration (e.g., six 21-day cycles) or involving the administration of less than 20 mg/dose (e.g., 10 mg per dose) is referred to herein as a “reduced” human dosing regimen. Examples of reduced human dosing regimens include the following: (i) 15 mg/dose, 12.5 mg/dose, 10 mg/dose, 7.5 mg/dose, 5 mg/dose, 4 mg/dose, 3 mg/dose, 2 mg/dose, 1 mg/dose, and 0.5 mg/dose; (ii)<20 mg/dose, 12.5-15 mg/dose, 10-12.5 mg/dose, 7.5-10 mg/dose, 5-7.5 mg/dose, 4-5 mg/dose, 3-4 mg/dose, 2-3 mg/dose, 1-2 mg/dose, 0.5-1 mg/dose, and <0.5 mg/dose; and (iii) dosing on days 1, 3, 5, 8, and 10 of a 21-day cycle, dosing on days 1, 3, 5, 8, and 12 of a 21-day cycle, dosing on days 1, 3, 8, 10, and 12 of a 21-day cycle, dosing on days 1, 5, 8, 10, and 12 of a 21-day cycle, dosing on days 1, 5, 8, and 12 of a 21-day cycle, dosing on days 1 and 8 of a 21-day cycle, dosing on day 1 of a 21-day cycle, dosing on day 1 of a 14-day cycle, and dosing on day 1 of a 7-day cycle. In one embodiment, each of the normal and reduced human dosing regimens of panobinostat further includes the administration of bortezomib and dexamethasone as directed in the FDA label for panobinostat.

The terms “normal” human dosing regimen and “reduced” human dosing regimen also apply, mutatis mutandis, to any other HDAC inhibitor with respect to its approved or otherwise customary dosing regimen.

### Example 9—²²⁵Ac-HuM195—Normal and Reduced Dosing Regimens

For an agent such as an antibody labeled with an alpha-emitting isotope, the majority of the drug administered to a subject typically consists of non-labeled antibody, with the minority being the labeled antibody. Doses of labeled agent used in connection with this invention include, for example, a single administration, and two or more administrations (i.e., fractions). The amount administered in each dose can be measured, for example, by labeled radiation activity (e.g., μCi/kg) or antibody weight (e.g., μg/kg or μg/m²).

In the case of ²²⁵Ac-HuM195, the “normal” human dosing regimen (regardless of the disorder treated), as this term is used herein, includes either of the following: (i) 4.0 μCi/kg administered fractionally in multiple administrations over no less than 1 week apart between doses; or (ii) 4.0 μCi/kg when delivered in a single administration.

A dosing regimen involving the administration of less ²²⁵Ac-HuM195 (e.g., 2.0 μCi/kg when delivered in a single administration) is referred to herein as a “reduced” human dosing regimen. Additional reduced human dosing regimens include, for example: (i) 2×<0.25 μCi/kg, 2×0.25 μCi/kg, 2×<0.5 μCi/kg, 2×0.5 μCi/kg, 2×<0.75 μCi/kg, 2×0.75 μCi/kg, 2×<1.0 μCi/kg, 2×1.0 μCi/kg, 2×<1.25 μCi/kg, 2×1.25 μCi/kg, 2×<1.5 μCi/kg, or 2×1.5 μCi/kg, where the fractions are administered one week apart; or (ii) 0.25 μCi/kg, 0.5 μCi/kg, 0.75 μCi/kg, 1.0 μCi/kg, 1.25 μCi/kg, 1.5 μCi/kg, 1.75 μCi/kg, 2.0 μCi/kg, 2.5 μCi/kg, 3.0 μCi/kg or 3.5 μCi/kg when delivered in a single administration. As a further example, reduced human dosing regimens of ²²⁵Ac-HuM195 include those corresponding to 25%, 50% or 75% of the normal dosing regimen.

The terms “normal” human dosing regimen and “reduced” human dosing regimen also apply, mutatis mutandis, to any other alpha-emitting isotope-labeled agent with respect to its approved or otherwise customary dosing regimen.

### Example 10—Dosing Scenario I for ²²⁵Ac-HuM195 and One of Vorinostat, Romidepsin, Belinostat, or Panobinostat

A human AML patient is treated according to the following regimen. One of vorinostat, romidepsin, belinostat, or panobinostat (referred to in this Example as “HDACi”) is orally administered according to its normal dosing regimen, accompanied by intravenous administration of ²²⁵Ac-HuM195 according to its normal dosing regimen (either single or fractional administration). In this Example and the others where applicable, the dosing regimens include the following embodiments, by way of example: (a) the HDACi and antibody radioconjugate are administered concurrently, wherein (i) each is administered beginning on the same day, (ii) the antibody is administered in a single dose or fractionated doses not less than one week apart, and (iii) the HDACi is administered daily or less frequently (as appropriate), and for a duration equal to or exceeding that of the antibody administration; or (b) the HDACi and antibody radioconjugate are administered concurrently, wherein (i) the HDACi administration precedes antibody radioconjugate administration by at least one week, (ii) the antibody is administered in a single dose or fractionated doses not less than one week apart, and (iii) the HDACi is administered daily or less frequently (as appropriate), and for a duration equal to or exceeding that of the antibody administration.

Also envisioned is the treatment of an experimental mouse model according to the treatment regimen in this scenario, whereby the appropriate dosing regimens are commensurate with mouse body weight and tumor xenograft size.

### Example 11—Dosing Scenario II for ²²⁵Ac-HuM195 and One of Vorinostat, Romidepsin, Belinostat, or Panobinostat

A human AML patient is treated according to the following regimen. One of vorinostat, romidepsin, belinostat, or panobinostat (referred to in this Example as “HDACi”) is orally administered according to its normal dosing regimen, accompanied by intravenous administration of ²²⁵Ac-HuM195 according to a reduced dosing regimen (either single or fractional administration). In one embodiment, the reduced dosing regimen of ²²⁵Ac-HuM195 is (i) 2×0.5 μCi/kg, 2 10×1.0 μCi/kg, or 2×1.5 μCi/kg, where the fractions are administered one week apart; or (ii) 1×0.5 μCi/kg, 1×1.0 μCi/kg, 1×2.0 μCi/kg, or 1×3.0 μCi/kg, for a single administration. In another embodiment, the reduced human dosing regimen of HDACi includes that corresponding to 25%, 50% or 75% of its respective normal dosing regimen.

Also envisioned is the treatment of an experimental mouse model according to the treatment regimen in this scenario, whereby the appropriate dosing regimens are commensurate with mouse body weight and tumor xenograft size.

### Example 12—Dosing Scenario III for ²²⁵Ac-HuM195 and One of Vorinostat, Romidepsin, Belinostat, or Panobinostat

A human AML patient is treated according to the following regimen. One of vorinostat, romidepsin, belinostat, or panobinostat (referred to in this Example as “HDACi”) is orally administered according to a reduced dosing regimen, accompanied by intravenous administration of the normal dosing regimen of ²²⁵Ac-HuM195 (either single or fractional administration). In one embodiment, the reduced dosing regimen of HDACi is one of the following: (i) where the HDACi is vorinostat, the reduced dosing regimen is 350 mg/day, 300 mg/day, 250 mg/day, 200 mg/day, 150 mg/day, 100 mg/day, 75 mg/day, 50 mg/day, or 25 mg/day; (ii) where the HDACi is romidepsin, the reduced dosing regimen is 13 mg/m² per administration, 12 mg/m² per administration, 11 mg/m² per administration, 10 mg/m² per administration, 9 mg/m² per administration, 8 mg/m² per administration, 7 mg/m² per administration, 6 mg/m² per administration, 5 mg/m² per administration, 4 mg/m² per administration, 3 mg/m² per administration, 2 mg/m² per administration, 1 mg/m² per administration, or 0.5 mg/m² per administration; (iii) where the HDACi is belinostat, the reduced dosing regimen is 900 mg/m² per administration, 800 mg/m² per administration, 700 mg/m² per administration, 600 mg/m² per administration, 500 mg/m² per administration, 400 mg/m² per administration, 300 mg/m² per administration, 250 mg/m² per administration, 200 mg/m² per administration, 150 mg/m² per administration, 100 mg/m² per administration, 75 mg/m² per administration, 50 mg/m² per administration, or 25 mg/m² per administration; and (iv) where the HDACi is panobinostat, the reduced dosing regimen is 15 mg/dose, 12.5 mg/dose, 10 mg/dose, 7.5 mg/dose, 5 mg/dose, 4 mg/dose, 3 mg/dose, 2 mg/dose, 1 mg/dose, or 0.5 mg/dose. In another embodiment, the reduced human dosing regimen of vorinostat, romidepsin, belinostat, and panobinostat includes that corresponding to 25%, 50% or 75% of its respective normal dosing regimen.

Also envisioned is the treatment of an experimental mouse model according to the treatment regimen in this scenario, whereby the appropriate dosing regimens are commensurate with mouse body weight and tumor xenograft size.

### Example 13—Dosing Scenario IV for ²²⁵Ac-HuM195 and One of Vorinostat, Romidepsin, Belinostat, or Panobinostat

A human AML patient is treated according to the following regimen. One of vorinostat, romidepsin, belinostat, or panobinostat (referred to in this Example as “HDACi”) is orally administered according to a reduced dosing regimen, accompanied by intravenous administration of a reduced dosing regimen of ²²⁵Ac-HuM195 (either single or fractional administration). In one embodiment, (a) the reduced dosing regimen of ²²⁵Ac-HuM195 is one of (i) 2×0.5 μCi/kg, 2×1.0 μCi/kg, or 2×1.5 μCi/kg, where the fractions are administered one week apart; or (ii) 1×0.5 μCi/kg, 1×1.0 μCi/kg, 1×2.0 μCi/kg, or 1×3.0 μCi/kg, for a single administration, and (b) the reduced dosing regimen of HDACi is one of the following: (i) where the HDACi is vorinostat, the reduced dosing regimen is 350 mg/day, 300 mg/day, 250 mg/day, 200 mg/day, 150 mg/day, 100 mg/day, 75 mg/day, 50 mg/day, or 25 mg/day; (ii) where the HDACi is romidepsin, the reduced dosing regimen is 13 mg/m² per administration, 12 mg/m² per administration, 11 mg/m² per administration, 10 mg/m² per administration, 9 mg/m² per administration, 8 mg/m² per administration, 7 mg/m² per administration, 6 mg/m² per administration, 5 mg/m² per administration, 4 mg/m² per administration, 3 mg/m² per administration, 2 mg/m² per administration, 1 mg/m² per administration, or 0.5 mg/m² per administration; (iii) where the HDACi is belinostat, the reduced dosing regimen is 900 mg/m² per administration, 800 mg/m² per administration, 700 mg/m² per administration, 600 mg/m² per administration, 500 mg/m² per administration, 400 mg/m² per administration, 300 mg/m² per administration, 250 mg/m² per administration, 200 mg/m² per administration, 150 mg/m² per administration, 100 mg/m² per administration, 75 mg/m² per administration, 50 mg/m² per administration, or 25 mg/m² per administration; and (iv) where the HDACi is panobinostat, the reduced dosing regimen is 15 mg/dose, 12.5 mg/dose, 10 mg/dose, 7.5 mg/dose, 5 mg/dose, 4 mg/dose, 3 mg/dose, 2 mg/dose, 1 mg/dose, or 0.5 mg/dose. In another embodiment, the reduced human dosing regimen of vorinostat, romidepsin, belinostat, and panobinostat includes that corresponding to 25%, 50% or 75% of its respective normal dosing regimen.

Also envisioned is the treatment of an experimental mouse model according to the treatment regimen in this scenario, whereby the appropriate dosing regimens are commensurate with mouse body weight and tumor xenograft size.

### Example 14—Dosing Scenarios for ²²⁵Ac-Labeled Anti-CD38 mAb and Vorinostat Or Romidepsin in Treating CTCL

Envisioned in this invention is an embodiment of the present therapeutic method wherein the hematologic malignancy is cutaneous T-cell lymphoma (CTCL), the radioisotope-labeled agent is an anti-CD38 antibody, such as Daratumumab and Isatuximab, and the HDAC inhibitor is vorinostat or romidepsin. A human CTCL patient may be treated according to one of the following two regimens.

In the first regimen, vorinostat is orally administered according to a reduced dosing regimen, accompanied by intravenous administration of a reduced dosing regimen of ²²⁵Ac-Daratumumab or ²²⁵Ac Isatuximab (either single or fractional administration). In one embodiment, (a) the reduced dosing regimen of ²²⁵Ac-labeled antibody is one of (i) 2×0.5 μCi/kg, 2×1.0 μCi/kg, or 2×1.5 μCi/kg, where the fractions are administered one week apart; or (ii) 1×0.5 μCi/kg, 1×1.0 μCi/kg, 1×2.0 μCi/kg, or 1×3.0 μCi/kg, for a single administration, and (b) the reduced dosing regimen of vorinostat is 350 mg/day, 300 mg/day, 250 mg/day, 200 mg/day, 150 mg/day, 100 mg/day, 75 mg/day, 50 mg/day, or 25 mg/day. In another embodiment, the reduced human dosing regimen of vorinostat includes that corresponding to 25%, 50% or 75% of its respective normal dosing regimen.

In the second regimen, romidepsin is intravenously administered according to a reduced dosing regimen, accompanied by intravenous administration of a reduced dosing regimen of the ²²⁵Ac-labeled antibody (either single or fractional administration). In one embodiment, (a) the reduced dosing regimen of the ²²⁵Ac-labeled antibody is one of (i) 2×0.5 μCi/kg, 2×1.0 μCi/kg, or 2×1.5 μCi/kg, where the fractions are administered one week apart; or (ii) 1×0.5 μCi/kg, 1×1.0 μCi/kg, 1×2.0 μCi/kg, or 1×3.0 μCi/kg, for a single administration, and (b) the reduced dosing regimen of romidepsin is 13 mg/m² per administration, 12 mg/m² per administration, 11 mg/m² per administration, 10 mg/m² per administration, 9 mg/m² per administration, 8 mg/m² per administration, 7 mg/m² per administration, 6 mg/m² per administration, 5 mg/m² per administration, 4 mg/m² per administration, 3 mg/m² per administration, 2 mg/m² per administration, 1 mg/m² per administration, or 0.5 mg/m² per administration. In another embodiment, the reduced human dosing regimen of romidepsin includes that corresponding to 25%, 50% or 75% of its respective normal dosing regimen.

Also envisioned is the treatment of an experimental mouse model according to the treatment regimens in this scenario, whereby the appropriate dosing regimens are commensurate with mouse body weight and tumor xenograft size.

### Example 15—Dosing Scenario for ²²⁵Ac-Labeled Anti-CD38 mAb and Belinostat in Treating PTCL

Envisioned in this invention is an embodiment of the present therapeutic method wherein the hematologic malignancy is peripheral T-cell lymphoma (PTCL), the radioisotope-labeled agent is an anti-CD38 antibody, and the HDAC inhibitor is belinostat. A human PTCL patient may be treated according to the following regimen. Belinostat is intravenously administered according to a reduced dosing regimen, accompanied by intravenous administration of a reduced dosing regimen of ²²⁵Ac-Daratumumab or ²²⁵Ac Isatuximab (either single or fractional administration). In one embodiment, (a) the reduced dosing regimen of ²²⁵Ac-labeled antibody is one of (i) 2×0.5 μCi/kg, 2×1.0 μCi/kg, or 2×1.5 μCi/kg, where the fractions are administered one week apart; or (ii) 1×0.5 μCi/kg, 1×1.0 μCi/kg, 1×2.0 μCi/kg, or 1×3.0 μCi/kg, for a single administration, and (b) the reduced dosing regimen of belinostat is 900 mg/m² per administration, 800 mg/m² per administration, 700 mg/m² per administration, 600 mg/m² per administration, 500 mg/m² per administration, 400 mg/m² per administration, 300 mg/m² per administration, 250 mg/m² per administration, 200 mg/m² per administration, 150 mg/m² per administration, 100 mg/m² per administration, 75 mg/m² per administration, 50 mg/m² per administration, or 25 mg/m² per administration. In another embodiment, the reduced human dosing regimen of belinostat includes that corresponding to 25%, 50% or 75% of its respective normal dosing regimen.

Also envisioned is the treatment of an experimental mouse model according to the treatment regimen in this scenario, whereby the appropriate dosing regimens are commensurate with mouse body weight and tumor xenograft size.

Any and all publications, patents, patent applications and other documents cited in this application are hereby incorporated by reference in their entireties for all purposes to the same extent as if each individual publication, patent, patent application or other document were individually indicated to be incorporated by reference for all purposes.

It should be understood that wherever in this disclosure an embodiment of the invention or an element or step thereof is described in terms of “including,” “include(s),” “comprising,” or “comprise(s),” corresponding embodiments, elements or steps thereof expressed, instead, in terms of “consisting essentially of” or “consisting of” are also intended to be disclosed and provided by this disclosure.

While various specific embodiments have been illustrated and described herein, it will be appreciated that various changes can be made without departing from the spirit and scope of the invention(s). Moreover, features described in connection with one embodiment of the invention may be used in conjunction with other embodiments, even if not explicitly exemplified in combination within.

### REFERENCES

1. Gansow, et al., U.S. Pat. No. 4,923,985.
2. Scheinberg, et al., U.S. Pat. No. 6,683,162.
3. Brandwein, et al., 2007. Leukemia. 21:821-4.
4. Co, et al., J. Immunol. 148:1149-1154, 1992.
5. Eckschlager, et al., Int. J. Mol. Sci. 2017, 18, 1414.
6. McDevitt, Applied Radiation and Isotope, 57 (2002), 841-847.

What is claimed is:

1. A method for treating a human subject afflicted with acute myeloid leukemia, comprising administering to the subject (i) one or both of a histone deacetylase (HDAC) inhibitor and an LSD1 inhibitor, and (ii) ²²⁵Ac-labeled HuM195, wherein the amounts of the HDAC inhibitor and/or LSD1 inhibitor, and ²²⁵Ac-labeled HuM195, when administered in conjunction with one another, are therapeutically effective.

2. The method of claim 1, wherein the administering step comprises:
administering the HDAC inhibitor to the subject.

3. The method of claim 1 or 2, wherein the administering step comprises:
administering the LSD1 inhibitor to the subject.

4. A method for treating a mammalian subject afflicted with cancer, comprising administering to the subject (i) one or both of a histone deacetylase (HDAC) inhibitor and an LSD1 inhibitor, and (ii) a radioisotope-labeled agent that targets cancer cells in the subject, wherein the amounts of the HDAC inhibitor and/or the LSD1 inhibitor, and radioisotope-labeled agent, when administered in conjunction with one another, are therapeutically effective.

5. The method of claim 4, wherein the subject is human.

6. The method of claim 4 or 5, wherein the cancer is a solid tumor.

7. The method of any one of claims 4-6, wherein the cancer is selected from the group consisting of breast cancer, ovarian cancer, prostate cancer, lung cancer, squamous cell carcinoma of the head and neck, gastric cancer, pancreatic cancer, brain cancer, liver cancer, sarcoma and melanoma.

8. The method of any one of claims 4-7, wherein the cancer is selected from the group consisting of breast cancer and ovarian cancer.

9. The method of claim 4 or 5, wherein the cancer is a hematologic malignancy.

10. The method of any one of claims 4, 5 and 9, wherein the cancer is selected from the group consisting of acute myeloid leukemia, myelodysplastic syndrome, and multiple myeloma.

11. The method of claim 10, wherein the hematologic malignancy is acute myeloid leukemia.

12. The method of any of claims 4-11, wherein the HDAC inhibitor is selected from the group consisting of vorinostat, romidepsin, belinostat, and panobinostat.

13. The method of any of claims 4-12, wherein the radioisotope-labeled agent is an antibody that specifically binds to a moiety selected from the group consisting of CD33, CD45, CD38, Her3, DR5 (TRAIL-R2), 5T4, Lewis-Y, CAIX, Her2, Claudin 18.2, CEACAM5, MUC1, and mesothelin.

14. The method of claim 13, wherein the antibody is selected from the group consisting of alemtuzumab, ibritumomab, brentuximab, trastuzumab, trastuzumab emtansine, gemtuzumab, lintuzumab, B1836858, BC8/apamistamab, daratumumab, felzartamab/MOR202, isatuximab/SAR650984, TAK-169, AV-203, CDX-3379, HMBD 001, patritumab, seribantumab, elgemtumab/LJM716, lumretuzumab/RG7116/RO5479599, GSK2849330, mapatumumab, tigatuzumab/CS-1008, drozitumab, lexatumumab, conatumumab, MED10641, PF-06263507/A1 mcMMAF, Anti-5T4 SYD1875, ASN004, AVA-020, Tb535, Hu3S193, B3, IGN311, BR96/BMS-182248-01, girentuximab, BAY 794620, BAY 2701439, MEDI-4276, XMT-1522, zolbetuximab, TST001, A315, labetuzumab, SAR566658, DMOT4039, anetumab, BAY 2287411, and amatuximab.

15. The method of any one of claims 4, 5, and 9-12, wherein the radioisotope-labeled agent is an anti-CD33 antibody labeled with an isotope selected from the group consisting of ⁹⁰Y, ⁸⁹Sr, ¹⁵³Sm, ³²P, ²²⁵Ac, ²¹³Bi, ²¹³Po ²¹¹At, ²¹²Bi, ²¹³Bi, ²²³Ra, ²²⁷Th, ¹⁴⁹Tb, ¹³¹I, ¹³⁷Cs, ²¹²Pb, ¹⁰³Pd, ¹⁶⁶Ho, ¹⁸⁶Re, ¹⁸⁸Re, ⁶⁷Cu, ¹⁹⁹Au, ¹⁰⁵Rh, ²¹¹As, and ¹⁷⁷Lu.

16. The method of claim 15, wherein the radioisotope-labeled agent is ²²⁵Ac-labeled HuM195 or ¹⁷⁷Lu-labeled HuM195.

17. A method for inducing the death of a mammalian cancer cell, comprising contacting the cell with (i) one or both of a histone deacetylase (HDAC) inhibitor and an LSD1 inhibitor, in conjunction with (ii) a radioisotope-labeled agent that targets the cancer cell, wherein the amounts of HDAC inhibitor and/or LSD1 inhibitor, and radiolabeled agent, when contacted with the cell in conjunction with one another, are effective to induce the cell's death.

18. The method of claim 17, wherein the cancer cell is a human cancer cell.

19. The method of claim 17 or 18, wherein the cancer cell is selected from the group consisting of a breast cancer cell, an ovarian cancer cell, a prostate cancer cell, a lung cancer cell, a squamous cell carcinoma of the head and neck cell, a gastric cancer cell, a pancreatic cancer cell, a brain cancer cell, a liver cancer cell, a sarcoma cell, and a melanoma cell.

20. The method of any of claims 17-19, wherein the cancer cell is selected from the group consisting of a breast cancer cell and an ovarian cancer cell.

21. The method of claim 17 or 19, wherein the cancer cell is a hematologic cancer cell.

22. The method of any of claims 17, 18, and 21, wherein the hematologic cancer cell is an acute myeloid leukemia cell, a myelodysplastic syndrome cell, or a multiple myeloma cell.

23. The method of any of claims 17, 18, 21, and 22, wherein the hematologic cancer cell is an acute myeloid leukemia cell.

24. The method of any of claims 17-23, wherein the HDAC inhibitor is selected from the group consisting of vorinostat, romidepsin, belinostat, and panobinostat.

25. The method of any of claims 17-23, wherein the radioisotope-labeled agent is an antibody that specifically binds to an antigen selected from the group consisting of CD33, CD45, CD38, Her3, DR5 (TRAIL-R2), 5T4, Lewis-Y, CAIX, Her2, Claudin 18.2, CEACAM5, MUC1, and mesothelin.

26. The method of claim 25, wherein the antibody is selected from the group consisting of alemtuzumab, ibritumomab, brentuximab, trastuzumab, gemtuzumab, lintuzumab, B1836858, BC8/apamistamab, daratumumab, felzartamab/MOR202, isatuximab/SAR650984, TAK-169, AV-203, CDX-3379, HMBD 001, patritumab, seribantumab, elgemtumab/LJM716, lumretuzumab/RG7116/RO5479599, GSK2849330, mapatumumab, tigatuzumab/CS-1008, drozitumab, lexatumumab, conatumumab, MED10641, PF-06263507/A1 mcMMAF, Anti-5T4 SYD1875, ASN004, AVA-020, Tb535, Hu3S193, B3, IGN311, BR96/BMS-182248-01, girentuximab, BAY 794620, BAY 2701439, MEDI-4276, XMT-1522, zolbetuximab, TST001, A315, labetuzumab, SAR566658, DMOT4039, anetumab, BAY 2287411, and amatuximab.

27. The method of any of claims 17-19 and 21-24, wherein the radioisotope-labeled agent is an anti-CD33 antibody labeled with a radioisotope selected from the group consisting of ⁹⁰Y, ⁸⁹Sr, ¹⁵³Sm, ³²P, ²²⁵Ac, ²¹³Bi, ²¹³Po, ²¹¹At, ²¹²Bi, ²¹³Bi, ²²³Ra, ²²⁷Th, ¹⁴⁹Tb, ¹³¹I, ¹³⁷Cs, ²¹²Pb, ¹⁰³Pd, ¹⁶⁶Ho, ¹⁸⁶Re, ¹⁸⁸Re, ⁶⁷Cu, ¹⁹⁹Au, ¹⁰⁵Rh, ²¹¹As, and ¹⁷⁷Lu.

28. The method of any of claims 17-19 and 21-24, wherein the radioisotope-labeled agent is ²²⁵Ac-labeled HuM195.

29. A method for inducing the death of a mammalian acute myeloid leukemic cell, comprising contacting the cell with (i) one or both of a histone deacetylase (HDAC) inhibitor and an LSD1 inhibitor, and (ii) ²²⁵Ac-labeled HuM195, wherein the amounts of HDAC inhibitor and/or LSD1 inhibitor, and ²²⁵Ac-labeled HuM195, when contacted with the cell in conjunction with one another, are effective to induce the cell's death.

30. The method of claim 29, wherein the contacting step comprises:
contacting the cell with the HDAC inhibitor.

31. The method of claim 29 or 30, wherein the contacting step comprises:
contacting the cell with the LSD1 inhibitor.

32. Combination use of a cancer targeting agent labeled with an alpha-particle emitting radionuclide and one or both of an HDAC inhibitor and an LSD1 inhibitor for the treatment of cancer in a mammalian subject.

33. Combination use of a cancer targeting agent labeled with a beta-particle emitting radionuclide and one or both of an HDAC inhibitor and an LSD1 inhibitor for the treatment of cancer in a mammalian subject.


SEQUENCE LISTING (117 sequences)

SEQ ID NO: 1 - protein, 5 aa, Mus musculus
SHWLH

SEQ ID NO: 2 - protein, 17 aa, Mus musculus
VLDPSDFYSNYNQNFKG

SEQ ID NO: 3 - protein, 11 aa, Mus musculus
GLLSGDYAMDY

SEQ ID NO: 4 - protein, 16 aa, Mus musculus
RSSQSIVHSNGNTYLE

SEQ ID NO: 5 - protein, 7 aa, Mus musculus
KVSNRFS

SEQ ID NO: 6 - protein, 9 aa, Mus musculus
FQGSYVPWT

SEQ ID NO: 7 - protein, 120 aa, Mus musculus
QVQLQQPGAELVRPGTSVKLSCKASGYTFTSHWLHWVKQRPGQGLEWIGVLDPSDFYSNYNQNFKGKATLTVDTSSSTAYMQLSSLTSEDSAVYYCARGLLSGDYAMDYWGAGTSVTVSS

SEQ ID NO: 8 - protein, 112 aa, Mus musculus
DVLMTQIPLSLPVSLGDQASISCRSSQSIVHSNGNTYLEWYLQKPGQSPKSLIYKVSNRFSGVPDRFSGSGSGTDFTLKISRVEAEDLGVYYCFQGSYVPWTFGGGTKLEIK

SEQ ID NO: 9 - protein, 463 aa, Mus musculus
MGWSCIIVLLVSTATGVHSQVQLQQPGAELVRPGTSVKLSCKASGYTFTSHWLHWVKQRPGQGLEWIGVLDPSDFYSNYNQNFKGKATLTVDTSSSTAYMQLSSLTSEDSAVYYCARGLLSGDYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 10 - protein, 238 aa, Mus musculus
MKLPVRLLVLMFWIPASSSDVLMTQIPLSLPVSLGDQASISCRSSQSIVHSNGNTYLEWYLQKPGQSPKSLIYKVSNRFSGVPDRFSGSGSGTDFTLKISRVEAEDLGVYYCFQGSYVPWTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 11 - protein, 444 aa, Mus musculus
QVQLQQPGAELVRPGTSVKLSCKASGYTFTSHWLHWVKQRPGQGLEWIGVLDPSDFYSNYNQNFKGKATLTVDTSSSTAYMQLSSLTSEDSAVYYCARGLLSGDYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 12 - protein, 219 aa, Mus musculus
DVLMTQIPLSLPVSLGDQASISCRSSQSIVHSNGNTYLEWYLQKPGQSPKSLIYKVSNRFSGVPDRFSGSGSGTDFTLKISRVEAEDLGVYYCFQGSYVPWTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 13 - protein, 10 aa, Mus musculus
QVQLQQPGAE

SEQ ID NO: 14 - protein, 10 aa, Mus musculus
DVLMTQIPLS

SEQ ID NO: 15 - protein, 5 aa, Artificial Sequence
  Description of Artificial Sequence Synthetic peptide
DYAMS

SEQ ID NO: 16 - protein, 17 aa, Artificial Sequence
  Description of Artificial Sequence Synthetic peptide
TISDGGTYTYYPDSVKG

SEQ ID NO: 17 - protein, 10 aa, Artificial Sequence
  Description of Artificial Sequence Synthetic peptide
EWGDYDGFDY

SEQ ID NO: 18 - protein, 11 aa, Artificial Sequence
  Description of Artificial Sequence Synthetic peptide
RASQEISGYLS

SEQ ID NO: 19 - protein, 7 aa, Artificial Sequence
  Description of Artificial Sequence Synthetic peptide
AASTLDS

SEQ ID NO: 20 - protein, 9 aa, Artificial Sequence
  Description of Artificial Sequence Synthetic peptide
LQYDSYPYT

SEQ ID NO: 21 - protein, 119 aa, Artificial Sequence
  Description of Artificial Sequence Synthetic polypeptide
QVQLVESGGGLVKPGGSLRLSCAASGFTFSDYAMSWIRQAPGKGLEWVSTISDGGTYTYYPDSVKGRFTISRDNAKNSLYLQMNSLRAEDTAVYYCAREWGDYDGFDYWGQGTLVTVSS

SEQ ID NO: 22 - protein, 107 aa, Artificial Sequence
  Description of Artificial Sequence Synthetic polypeptide
DIQMTQSPSSLSASVGDRVTITCRASQEISGYLSWYQQKPGKAPKRLIYAASTLDSGVPSRFSGSGSGTEFTLTISSLQPEDFATYYCLQYDSYPYTFGQGTKLEIK

SEQ ID NO: 23 - protein, 471 aa, Artificial Sequence
  Description of Artificial Sequence Synthetic polypeptide
MDMRVPAQLLGLLLLWLRGARCQVQLVESGGGLVKPGGSLRLSCAASGFTFSDYAMSWIRQAPGKGLEWVSTISDGGTYTYYPDSVKGRFTISRDNAKNSLYLQMNSLRAEDTAVYYCAREWGDYDGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 236 aa, Artificial Sequence
  Description of Artificial Sequence Synthetic polypeptide
MDMRVPAQLLGLLLLWLRGARCDIQMTQSPSSLSASVGDRVTITCRASQEISGYLSWYQQKPGKAPKRLIYAASTLDSGVPSRFSGSGSGTEFTLTISSLQPEDFATYYCLQYDSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 5 aa, Artificial Sequence
  Description of Artificial Sequence Synthetic peptide
SHWLH

SEQ ID NO: 26 - protein, 17 aa, Artificial Sequence
  Description of Artificial Sequence Synthetic peptide
VLDPSDFYSNYNQNFKG

SEQ ID NO: 27 - protein, 11 aa, Artificial Sequence
  Description of Artificial Sequence Synthetic peptide
GLLSGDYAMDY

SEQ ID NO: 28 - protein, 16 aa, Artificial Sequence
  Description of Artificial Sequence Synthetic peptide
RSSQSIVHSNGNTYLE

SEQ ID NO: 29 - protein, 7 aa, Artificial Sequence
  Description of Artificial Sequence Synthetic peptide
KVSNRFS

SEQ ID NO: 30 - protein, 9 aa, Artificial Sequence
  Description of Artificial Sequence Synthetic peptide
FQGSYVPWT

SEQ ID NO: 31 - protein, 120 aa, Artificial Sequence
  Description of Artificial Sequence Synthetic polypeptide
QVQLQQPGAELVRPGTSVKLSCKASGYTFTSHWLHWVKQRPGQGLEWIGVLDPSDFYSNYNQNFKGKATLTVDTSSSTAYMQLSSLTSEDSAVYYCARGLLSGDYAMDYWGQGTSVTVSS

SEQ ID NO: 32 - protein, 112 aa, Artificial Sequence; identical to SEQ ID NO: 8
  Description of Artificial Sequence Synthetic polypeptide

SEQ ID NO: 33 - protein, 463 aa, Artificial Sequence; identical to SEQ ID NO: 9
  Description of Artificial Sequence Synthetic polypeptide

SEQ ID NO: 34 - protein, 238 aa, Artificial Sequence; identical to SEQ ID NO: 10
  Description of Artificial Sequence Synthetic polypeptide

SEQ ID NO: 35 - protein, 7 aa, Artificial Sequence
  Description of Artificial Sequence Synthetic peptide
TFGLSVG

SEQ ID NO: 36 - protein, 16 aa, Artificial Sequence
  Description of Artificial Sequence Synthetic peptide
HIWWDDDKYYNPALKS

SEQ ID NO: 37 - protein, 10 aa, Artificial Sequence
  Description of Artificial Sequence Synthetic peptide
IGADALPFDY

SEQ ID NO: 38 - protein, 16 aa, Artificial Sequence
  Description of Artificial Sequence Synthetic peptide
RSSKSLLHSNGNTYLY

SEQ ID NO: 39 - protein, 7 aa, Artificial Sequence
  Description of Artificial Sequence Synthetic peptide
RMSNLAS

SEQ ID NO: 40 - protein, 9 aa, Artificial Sequence
  Description of Artificial Sequence Synthetic peptide
MQHLEYPFT

SEQ ID NO: 41 - protein, 120 aa, Artificial Sequence
  Description of Artificial Sequence Synthetic polypeptide
QVTLKESGPGILRPSQTLSLTCSFSGFSLSTFGLSVGWIRQPSGKGLEWLAHIWWDDDKYYNPALKSRLTISKDTSKNQVFLKIANVDTADTATYYCARIGADALPFDYWGQGTTLTVSS

SEQ ID NO: 42 - protein, 112 aa, Artificial Sequence
  Description of Artificial Sequence Synthetic polypeptide
DIVLTQTAPSVPVTPGESVSISCRSSKSLLHSNGNTYLYWFLQRPGQSPQLLIYRMSNLASGVPDRFSGSGSGTAFTLRISRVEAEDVGVYYCMQHLEYPFTFGSGTKLEIK

SEQ ID NO: 43 - protein, 475 aa, Artificial Sequence
  Description of Artificial Sequence Synthetic polypeptide
MGRLTSSFLLLIVPAYVLSQVTLKESGPGILRPSQTLSLTCSFSGFSLSTFGLSVGWIRQPSGKGLEWLAHIWWDDDKYYNPALKSRLTISKDTSKNQVFLKIANVDTADTATYYCARIGADALPFDYWGQGTTLTVSSAKTTPPSVYPLAPGCGDTTGSSVTSGCLVKGYFPEPVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYTLPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 44 - protein, 239 aa, Artificial Sequence
  Description of Artificial Sequence Synthetic polypeptide
MRCLAEFLGLLVLWIPGAIGDIVLTQTAPSVPVTPGESVSISCRSSKSLLHSNGNTYLYWFLQRPGQSPQLLIYRMSNLASGVPDRFSGSGSGTAFTLRISRVEAEDVGVYYCMQHLEYPFTFGSGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 45 - protein, 5 aa, Artificial Sequence
  Description of Artificial Sequence Synthetic peptide
DHIIH

SEQ ID NO: 46 - protein, 17 aa, Artificial Sequence
  Description of Artificial Sequence Synthetic peptide
YIYPRDGYIKYNEKFKG

SEQ ID NO: 47 - protein, 8 aa, Artificial Sequence
  Description of Artificial Sequence Synthetic peptide
GYYYAMDY

SEQ ID NO: 48 - protein, 16 aa, Artificial Sequence
  Description of Artificial Sequence Synthetic peptide
RSSQSIVHSIGNTYLE

SEQ ID NO: 49 - protein, 9 aa, Artificial Sequence
  Description of Artificial Sequence Synthetic peptide
FQGSHVPFT

SEQ ID NO: 50 - protein, 117 aa, Artificial Sequence
  Description of Artificial Sequence Synthetic polypeptide
QVQLQQSDAELVKPGASVKISCKVSGYTFTDHIIHWMKQRPEQGLEWIGYIYPRDGYIKYNEKFKGKATLTADKSSSTAYMQVNSLTSEDSAVYFCARGYYYAMDYWGQGTSVTVSS

SEQ ID NO: 51 - protein, 112 aa, Artificial Sequence
  Description of Artificial Sequence Synthetic polypeptide
DVLMTQTPLSLPVSLGDQASISCRSSQSIVHSIGNTYLEWYLQKPGQSPKLLIYKVSNRFSGVPERFSGSGSGTDFTLKISRVEAEDLGVYYCFQGSHVPFTFGSGTKLEIK

SEQ ID NO: 52 - protein, 460 aa, Artificial Sequence
  Description of Artificial Sequence Synthetic polypeptide
MEWSWVSLFFLSVTTGVHSQVQLQQSDAELVKPGASVKISCKVSGYTFTDHIIHWMKQRPEQGLEWIGYIYPRDGYIKYNEKFKGKATLTADKSSSTAYMQVNSLTSEDSAVYFCARGYYYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 53 - protein, 238 aa, Artificial Sequence
  Description of Artificial Sequence Synthetic polypeptide
MKLPVRLLVLMFWIPASRSDVLMTQTPLSLPVSLGDQASISCRSSQSIVHSIGNTYLEWYLQKPGQSPKLLIYKVSNRFSGVPERFSGSGSGTDFTLKISRVEAEDLGVYYCFQGSHVPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 54 - protein, 5 aa, Artificial Sequence
  Description of Artificial Sequence Synthetic peptide
SYWMH

SEQ ID NO: 55 - protein, 17 aa, Artificial Sequence
  Description of Artificial Sequence Synthetic peptide
MIDPSDVYTNYNPKFKG

SEQ ID NO: 56 - protein, 6 aa, Artificial Sequence
  Description of Artificial Sequence Synthetic peptide
NYSGDY

SEQ ID NO: 57 - protein, 115 aa, Artificial Sequence
  Description of Artificial Sequence Synthetic polypeptide
QVQLLQPGAELVRPGTSVKLSCKTSGYTFSSYWMHWVKQRPGQGLEWIGMIDPSDVYTNYNPKFKGKATLTVDTSSSTAYMQLSSLTSEDSAVYYCARNYSGDYWGQGTTLTVSS

SEQ ID NO: 58 - protein, 112 aa, Artificial Sequence
  Description of Artificial Sequence Synthetic polypeptide
DVLMTQIPLSLPVSLGDQASISCRSSQSIVHSNGNTYLEWYLQKPGQSPKLLIYKVSNRFSGVPDRFSGSGSGTDFTLKISRVEAEDLGVYYCFQGSYVPWTFGGGTKLEIK

SEQ ID NO: 59 - protein, 458 aa, Artificial Sequence
  Description of Artificial Sequence Synthetic polypeptide
MGWSCIIVLLVSTATCVHSQVQLLQPGAELVRPGTSVKLSCKTSGYTFSSYWMHWVKQRPGQGLEWIGMIDPSDVYTNYNPKFKGKATLTVDTSSSTAYMQLSSLTSEDSAVYYCARNYSGDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 60 - protein, 238 aa, Artificial Sequence
  Description of Artificial Sequence Synthetic polypeptide
MKLPVRLLVLMFWIPASSSDVLMTQIPLSLPVSLGDQASISCRSSQSIVHSNGNTYLEWYLQKPGQSPKLLIYKVSNRFSGVPDRFSGSGSGTDFTLKISRVEAEDLGVYYCFQGSYVPWTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 61 - protein, 5 aa, Artificial Sequence
  Description of Artificial Sequence Synthetic peptide
TYGMS

SEQ ID NO: 62 - protein, 17 aa, Artificial Sequence
  Description of Artificial Sequence Synthetic peptide
WINTYSGVPTYADDFKG

SEQ ID NO: 63 - protein, 12 aa, Artificial Sequence
  Description of Artificial Sequence Synthetic peptide
GRDGYQVAWFAY

SEQ ID NO: 64 - protein, 11 aa, Artificial Sequence
  Description of Artificial Sequence Synthetic peptide
ITSTDIDDDMN

SEQ ID NO: 65 - protein, 7 aa, Artificial Sequence
  Description of Artificial Sequence Synthetic peptide
EGNTLRP

SEQ ID NO: 66 - protein, 9 aa, Artificial Sequence
  Description of Artificial Sequence Synthetic peptide
LQSDNLPYT

SEQ ID NO: 67 - protein, 121 aa, Artificial Sequence
  Description of Artificial Sequence Synthetic polypeptide
QIQLVQSGPELKKPGEAVKISCKSSGYTFTTYGMSWVKQAPGRALKWMGWINTYSGVPTYADDFKGRFAFSLESSASTAYLQINNLKNEDTATYFCARGRDGYQVAWFAYWGQGTLVTVSA

SEQ ID NO: 68 - protein, 107 aa, Artificial Sequence
  Description of Artificial Sequence Synthetic polypeptide
ETTVTQSPASLSMAIGDKVTIRCITSTDIDDDMNWFQQKPGEPPKLLISEGNTLRPGVPSRFSGSGYGTDFIFTIENMLSEDVADYYCLQSDNLPYTFGGGTKLEIK

SEQ ID NO: 69 - protein, 464 aa, Artificial Sequence
  Description of Artificial Sequence Synthetic polypeptide
MGWLWNLLFLMAAAQSAQAQIQLVQSGPELKKPGEAVKISCKSSGYTFTTYGMSWVKQAPGRALKWMGWINTYSGVPTYADDFKGRFAFSLESSASTAYLQINNLKNEDTATYFCARGRDGYQVAWFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 70 - protein, 234 aa, Artificial Sequence
  Description of Artificial Sequence Synthetic polypeptide
MFSLALLLSLLLLCVSDSRAETTVTQSPASLSMAIGDKVTIRCITSTDIDDDMNWFQQKPGEPPKLLISEGNTLRPGVPSRFSGSGYGTDFIFTIENMLSEDVADYYCLQSDNLPYTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 71 - protein, 5 aa, Artificial Sequence
  Description of Artificial Sequence Synthetic peptide
NYWMH

SEQ ID NO: 72 - protein, 17 aa, Artificial Sequence
  Description of Artificial Sequence Synthetic peptide
MIDPSDSYTNYNPKFKG

SEQ ID NO: 73 - protein, 115 aa, Artificial Sequence
  Description of Artificial Sequence Synthetic polypeptide
QVQLQQPGAELVRPGTSVKLSCKASGYTFTNYWMHWVKQRPGQGLEWIGMIDPSDSYTNYNPKFKGKATLTVDTSSSTAYMQLSSLTSEDSAVYYCARNYSGDYWGQGTTLTVSS

SEQ ID NO: 74 - protein, 112 aa, Artificial Sequence
  Description of Artificial Sequence Synthetic polypeptide
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSYVPWTFGGGTKLEIK

SEQ ID NO: 75 - protein, 458 aa, Artificial Sequence
  Description of Artificial Sequence Synthetic polypeptide
MGWSCIIVLLVSTATGVHSQVQLQQPGAELVRPGTSVKLSCKASGYTFTNYWMHWVKQRPGQGLEWIGMIDPSDSYTNYNPKFKGKATLTVDTSSSTAYMQLSSLTSEDSAVYYCARNYSGDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 76 - protein, 238 aa, Artificial Sequence
  Description of Artificial Sequence Synthetic polypeptide
MKLPVRLLVLMFWIPASSSDVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSYVPWTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 77 - protein, 449 aa, Artificial Sequence
  Description of Artificial Sequence Synthetic polypeptide
QVQLVESGGGLVKPGGSLRLSCAASGFTFSDYAMSWIRQAPGKGLEWVSTISDGGTYTYYPDSVKGRFTISRDNAKNSLYLQMNSLRAEDTAVYYCAREWGDYDGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 214 aa, Artificial Sequence
  Description of Artificial Sequence Synthetic polypeptide
DIQMTQSPSSLSASVGDRVTITCRASQEISGYLSWYQQKPGKAPKRLIYAASTLDSGVPSRFSGSGSGTEFTLTISSLQPEDFATYYCLQYDSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 119 aa, Artificial Sequence
  Description of Artificial Sequence Synthetic polypeptide
EVQLVESGGGLVQPGGSLRLSCAASGFTFSDYAMSWVRQAPGKGLEWVATISDGGTYTYYPDNVKGRFTISRDNAKNSLYLQMNSLRAEDTAVYYCAREWGDYDGFDYWGQGTLVTVSS

SEQ ID NO: 80 - protein, 119 aa, Artificial Sequence
  Description of Artificial Sequence Synthetic polypeptide
QVQLVESGGGLVKPGGSLRLSCAASGFTFSDYAMSWIRQAPGKGLEWVSTISDGGTYTYYPDNVKGRFTISRDNAKNSLYLQMNSLRAEDTAVYYCAREWGDYDGFDYWGQGTLVTVSS

SEQ ID NO: 81 - protein, 119 aa, Artificial Sequence; identical to SEQ ID NO: 21
  Description of Artificial Sequence Synthetic polypeptide

SEQ ID NO: 82 - protein, 119 aa, Artificial Sequence
  Description of Artificial Sequence Synthetic polypeptide
EVQLVESGGGLVKPGGSLRLSCAASGFTFSDYAMSWVRQAPGKGLEWVSTISDGGTYTYYPDNVKGRFTISRDNAKNSLYLQMNSLRAEDTAVYYCAREWGDYDGFDYWGQGTLVTVSS

SEQ ID NO: 83 - protein, 119 aa, Artificial Sequence
  Description of Artificial Sequence Synthetic polypeptide
EVQLLESGGGLVQPGGSLRLSCAASGFTFSDYAMSWVRQAPGKGLEWVSTISDGGTYTYYPDNVKGRFTISRDNSKNTLYLQMNSLRAEDTAVYYCAREWGDYDGFDYWGQGTLVTVSS

SEQ ID NO: 84 - protein, 119 aa, Artificial Sequence
  Description of Artificial Sequence Synthetic polypeptide
QVQLVESGGGVVQPGRSLRLSCAASGFTFSDYAMSWVRQAPGKGLEWVATISDGGTYTYYPDNVKGRFTISRDNSKNTLYLQMSSLRAEDTAVYYCAREWGDYDGFDYWGQGTLVTVSS

SEQ ID NO: 85 - protein, 119 aa, Artificial Sequence
  Description of Artificial Sequence Synthetic polypeptide
EVQLVESGGGLVKPGGSLRLSCAASGFTFSDYAMSWVRQAPGKGLEWVATISDGGTYTYYPDNVKGRFTISRDNAKNSLYLQMNSLRAEDTAVYYCAREWGDYDGFDYWGQGTLVTVSS

SEQ ID NO: 86 - protein, 107 aa, Artificial Sequence
  Description of Artificial Sequence Synthetic polypeptide
DIQLTQSPSFLSASVGDRVTITCRASQEISGYLSWYQQKPGKAPKLLIYAASTLDSGVPSRFSGSGSGTEFTLTISSLQPEDFATYYCLQYDSYPYTFGQGTKLEIK

SEQ ID NO: 87 - protein, 107 aa, Artificial Sequence
  Description of Artificial Sequence Synthetic polypeptide
DIQMTQSPSSLSASVGDRVTITCRASQEISGYLSWFQQKPGKAPKSLIYAASTLDSGVPSRFSGSGSGTDFTLTISSLQPEDFATYYCLQYDSYPYTFGQGTKLEIK

SEQ ID NO: 88 - protein, 107 aa, Artificial Sequence; identical to SEQ ID NO: 22
  Description of Artificial Sequence Synthetic polypeptide

SEQ ID NO: 89 - protein, 107 aa, Artificial Sequence
  Description of Artificial Sequence Synthetic polypeptide
DIQMTQSPSSLSASVGDRVTITCRASQEISGYLSWYQQKPGKAPKLLIYAASTLDSGVPSRFSGSGSGTDFTFTISSLQPEDIATYYCLQYDSYPYTFGQGTKLEIK

SEQ ID NO: 90 - protein, 107 aa, Artificial Sequence
  Description of Artificial Sequence Synthetic polypeptide
DIQMTQSPSSLSASVGDRVTITCRASQEISGYLSWYQQKPGKAPKLLIYAASTLDSGVPSRFSGSGSGTDFTLTISSLQPEDFATYYCLQYDSYPYTFGQGTKLEIK

SEQ ID NO: 91 - protein, 107 aa, Artificial Sequence
  Description of Artificial Sequence Synthetic polypeptide
DIQMTQSPSSLSASVGDRVTITCRASQEISGYLSWLQQKPGGAIKRLIYAASTLDSGVPSRFSGSGSGSDYTLTISSLQPEDFATYYCLQYDSYPYTFGQGTKLEIK

SEQ ID NO: 92 - protein, 468 aa, Artificial Sequence
  Description of Artificial Sequence Synthetic polypeptide
MNFGLSLMFLVLVLKGVQCEVQLVESGGGLVKPGGSLKLSCAASGFTFSDYAMSWVRQTPEKRLEWVATISDGGTYTYYPDNVKGRFTISRDNAKNNLYLQMSHLKSEDTAMYYCAREWGDYDGFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 236 aa, Artificial Sequence
  Description of Artificial Sequence Synthetic polypeptide
MDMRVPAHVFGFLLLWFPGTRCDIQMTQSPSSLSASLGERVSLTCRASQEISGYLSWLQQKPDGTIKRLIYAASTLDSGVPKRFSGSRSGSDYSLTIGSLESEDLADYYCLQYDSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 471 aa, Artificial Sequence; identical to SEQ ID NO: 23
  Description of Artificial Sequence Synthetic polypeptide

SEQ ID NO: 95 - protein, 467 aa, Artificial Sequence
  Description of Artificial Sequence Synthetic polypeptide
MDMRVPAQLLGLLLLWLRGARCQVQLVESGGGLVKPGGSLRLSCAASGFTFSDYAMSWIRQAPGKGLEWVSTISDGGTYTYYPDSVKGRFTISRDNAKNSLYLQMNSLRAEDTAVYYCAREWGDYDGFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 96 - protein, 236 aa, Artificial Sequence
  Description of Artificial Sequence Synthetic polypeptide
MDMRVPAQLLGLLLLWLRGARCDIQMTQSPSSLSASVGDRVTITCRASQEISGYLSWFQQKPGKAPKSLIYAASTLDSGVPSRFSGSGSGTDFTLTISSLQPEDFATYYCLQYDSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - protein, 236 aa, Artificial Sequence; identical to SEQ ID NO: 24
  Description of Artificial Sequence Synthetic polypeptide

SEQ ID NO: 98 - protein, 449 aa, Artificial Sequence
  Description of Artificial Sequence Synthetic polypeptide
QVQLVESGGGLVKPGGSLRLSCAASGFTFSDYAMSWIRQAPGKGLEWVSTISDGGTYTYYPDSVKGRFTISRDNAKNSLYLQMNSLRAEDTAVYYCAREWGDYDGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 445 aa, Artificial Sequence
  Description of Artificial Sequence Synthetic polypeptide
QVQLVESGGGLVKPGGSLRLSCAASGFTFSDYAMSWIRQAPGKGLEWVSTISDGGTYTYYPDSVKGRFTISRDNAKNSLYLQMNSLRAEDTAVYYCAREWGDYDGFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 100 - protein, 214 aa, Artificial Sequence
  Description of Artificial Sequence Synthetic polypeptide
DIQMTQSPSSLSASVGDRVTITCRASQEISGYLSWFQQKPGKAPKSLIYAASTLDSGVPSRFSGSGSGTDFTLTISSLQPEDFATYYCLQYDSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - protein, 214 aa, Artificial Sequence; identical to SEQ ID NO: 78
  Description of Artificial Sequence Synthetic polypeptide

SEQ ID NO: 102 - protein, 450 aa, Artificial Sequence
  Description of Artificial Sequence Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - protein, 214 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - protein, 448 aa, Artificial Sequence
  Description of Artificial Sequence Synthetic polypeptide
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 105 - protein, 214 aa, Artificial Sequence
  Description of Artificial Sequence Synthetic polypeptide
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - protein, 447 aa, Artificial Sequence
  Description of Artificial Sequence Synthetic polypeptide
QVQLQQWGAGLLKPSETLSLTCAVYGGSFSGYYWSWIRQPPGKGLEWIGEINHSGSTNYNPSLKSRVTISVETSKNQFSLKLSSVTAADTAVYYCARDKWTWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - protein, 220 aa, Artificial Sequence
  Description of Artificial Sequence Synthetic polypeptide
DIEMTQSPDSLAVSLGERATINCRSSQSVLYSSSNRNYLAWYQQNPGQPPKLLIYWASTRESGVPDRFSGSGSGTDFTLTISSLQAEDVAVYYCQQYYSTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - protein, 445 aa, Artificial Sequence
  Description of Artificial Sequence Synthetic polypeptide
EVQLLESGGGLVQPGGSLRLSCAASGFTFSHYVMAWVRQAPGKGLEWVSSISSSGGWTLYADSVKGRFTISRDNSKNTLYLQMNSLRAEDTAVYYCTRGLKMATIFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 109 - protein, 217 aa, Artificial Sequence
  Description of Artificial Sequence Synthetic polypeptide
QSALTQPASVSGSPGQSITISCTGTSSDVGSYNVVSWYQQHPGKAPKLIIYEVSQRPSGVSNRFSGSKSGNTASLTISGLQTEDEADYYCCSYAGSSIFVIFGGGTKVTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 110 - protein, 449 aa, Artificial Sequence
  Description of Artificial Sequence Synthetic polypeptide
QVQLVQSGAEVKKPGASVKVSCKASGYTFRSSYISWVRQAPGQGLEWMGWIYAGTGSPSYNQKLQGRVTMTTDTSTSTAYMELRSLRSDDTAVYYCARHRDYYSNSLTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 111 - protein, 220 aa, Artificial Sequence
  Description of Artificial Sequence Synthetic polypeptide
DIVMTQSPDSLAVSLGERATINCKSSQSVLNSGNQKNYLTWYQQKPGQPPKLLIYWASTRESGVPDRFSGSGSGTDFTLTISSLQAEDVAVYYCQSDYSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - protein, 446 aa, Artificial Sequence
  Description of Artificial Sequence Synthetic polypeptide
EVQLLESGGGLVQPGGSLRLSCAASGFTFSSYAMSWVRQAPGKGLEWVSAINSQGKSTYYADSVKGRFTISRDNSKNTLYLQMNSLRAEDTAVYYCARWGDEGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 113 - protein, 214 aa, Artificial Sequence
  Description of Artificial Sequence Synthetic polypeptide
DIQMTQSPSSLSASVGDRVTITCRASQGISNWLAWYQQKPGKAPKLLIYGASSLQSGVPSRFSGSGSGTDFTLTISSLQPEDFATYYCQQYSSFPTTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - DNA, 1952 bp, Artificial Sequence
  Description of Artificial Sequence Synthetic polynucleotide
tctagaccaccatggagaaagacacactcctgctatgggtcctacttctctgggttccaggttccacaggtgacattcagatgacccagtctccgagctctctgtccgcatcagtaggagacagggtcaccatcacatgcagagccagcgaaagtgtcgacaattatggcattagctttatgaactggttccaacagaaacccgggaaggctcctaagcttctgatttacgctgcatccaaccaaggctccggggtaccctctcgcttctcaggcagtggatctgggacagacttcactctcaccatttcatctctgcagcctgatgacttcgcaacctattactgtcagcaaagtaaggaggttccgtggacgttcggtcaagggaccaaggtggagatcaaacgtaagtagaatccaaagtctagaaattctaaactctgagggggtcggatgacgtggccattctttgcctaaagcattgagtttactgcaaggtcagaaaagcatgcaaagccctcagaatggctgcaaagagctccaacaaaacaatttagaactttattaaggaatagggggaagctaggaagaaactcaaaacatcaagattttaaatacgcttcttggtctccttgctataattatctgggataagcatgctgttttctgtctgtccctaacatgctctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagagggagaagtgcccccacctgctcctcagttccagcctgaccccctcccatcctttggcctctgaccctttttccacaggggacctacccctattgcggtcctccagctcatctttcacctcacccccctcctcctccttggctttaattatgctaatgttggaggagaatgaataaataaagtgaatctttgcacctgtggtttctctctttcctcatttaataattattatctgttgttttaccaactactcaatttctcttataagggactaaatatgtagtcatcctaaggcgcataaccatttataaaaatcatccttcattctattttaccctatcatcctctgcaagacagtcctccctcaaacccacaagccttctgtcctcacagtcccctgggccatggtaggagagacttgcttccttgttttcccctcctcagcaagccctcatagtcctttttaagggtgacaggtcttacagtcatatatcctttgattcaattccctgagaatcaaccaaagcaaatttttcaaaagaagaaacctgctataaagagaatcattcattgcaacatgatataaaataacaacacaataaaagcaattaaataaacaaacaatagggaaatgtttaagttcatcatggtacttagacttaatggaatgtcatgccttatttacatttttaaacaggtactgagggactcctgtctgccaagggccgtattgagtactttccacaacctaatttaatccacactatactgtgagattaaaaacattcattaaaatgttgcaaaggttctataaagctgagagacaaatatattctataactcagcaatcccacttctaggatcc

SEQ ID NO: 115 - protein, 238 aa, Artificial Sequence
  Description of Artificial Sequence Synthetic polypeptide
MEKDTLLLWVLLLWVPGSTGDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - DNA, 3246 bp, Artificial Sequence
  Description of Artificial Sequence Synthetic polynucleotide
tctagaccaccatgggatggagctggatctttctcttcctcctgtcaggaactgctggcgtccactctcaggttcagctggtgcagtctggagctgaggtgaagaagcctgggagctcagtgaaggtttcctgcaaagcttctggctacaccttcactgactacaacatgcactgggtgaggcaggctcctggccaaggcctggaatggattggatatatttatccttacaatggtggtaccggctacaaccagaagttcaagagcaaggccacaattacagcagacgagagtactaacacagcctacatggaactctccagcctgaggtctgaggacactgcagtctattactgcgcaagagggcgccccgctatggactactggggccaagggactctggtcactgtctcttcaggtaagaatggcctctagaccaccatgggatggagctttctggggcaggccaggcctgaccttggctttggggcagggagggggctaaggtgaggcaggtggcgccagccaggtgcacacccaatgcccatgagcccagacactggacgctgaacctcgcggacagttaagaacccaggggcctctgcgccctgggcccagctctgtcccacaccgcggtcacatggcaccacctctcttgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttggtgagaggccagcacagggagggagggtgtctgctggaagccaggctcagcgctcctgcctggacgcatcccggctatgcagccccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccggcaagcccccgctccccgggctctcgcggtcgcacgaggatgcttggcacgtaccccctgtacatacttcccgggcgcccagcatggaaataaagcacccagcgctgccctgggcccctgcgagactgtgatggttctttccacgggtcaggccgagtctgaggcctgagtggcatgagggaggcagagcgggtcccactgtccccacactggcccaggctgtgcaggtgtgcctgggccgcctagggtggggctcagccaggggctgccctcggcagggtgggggatttgccagcgtggccctccctccagcagcacctgccctgggctgggccacgggaagccctaggagcccctggggacagacacacagcccctgcctctgtaggagactgtcctgttctgtgagcgccctgtcctccgacctccatgcccactcgggggcatgcctagtccatgtgcgtagggacaggccctccctcacccatctacccccacggcactaacccctggctgccctgcccagcctcgcacccgcatggggacacaaccgactccggggacatgcactctcgggccctgtggagggactggtgcagatgcccacacacacactcagcccagacccgttcaacaaaccccgcactgaggttggccggccacacggccaccacacacacacgtgcacgcctcacacacggagcctcacccgggcgaactgcacagcacccagaccagagcaaggtcctcgcacacgtgaacactcctcggacacaggcccccacgagccccacgcggcacctcaaggcccacgagcctctcggcagcttctccacatgctgacctgctcagacaaacccagccctcctctcacaagggtgcccctgcagccgccacacacacacaggggatcacacaccacgtcacgtccctggccctggcccacttcccagtgccgcccttccctgcaggatcc

SEQ ID NO: 117 - protein, 465 aa, Artificial Sequence
  Description of Artificial Sequence Synthetic polypeptide
MGWSWIFLFLLSGTAGVHSQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK